SEQUENCE LISTING (11 sequences)

SEQ ID NO: 1 - DNA, 1957 bp, Homo sapiens
  CDS (83)...(1879)
ggtgggcgacgtggtccgggtcatcggcgaccttgacacagtgaagcggctgcaggctgggcatggcgagtggacggacgacatggcccctgccctgggccgcgtcgggaaggtggtgaaagtgtttggagacgggaacctgcgtgtagcagtcgctggtcagcggtggaccttcagcccctcctgcctggtggcctaccggcccgaggaggatgccaacctggacgtggccgagcgcgcccgggagaacaaaagctcactgagcgtggccctggacaagcttcgggcccagaagagtgacccagagcacccgggaaggctggtggtggaggtggcgctgggtaacgcagcccgggctctggacctgctgcggaggcgcccagagcaggtggacaccaagaaccaaggcaggaccgctctgcaagtggctgcctacctgggccaggtggagttgatacggctgctgctacaagccagggcgggcgtggacctgccggacgacgagggcaacacggcactgcactacgcggccctggggaaccagcccgaggccaccagggtgctcctgagtgctgggtgccgggcggacgccatcaacagcacccagagcacagcactgcacgtggccgtgcagaggggcttcctgaaggtggtgcgggccctgtgtgagcgcggctgtgacgtcaacctgcccgacgcccactcggacacgcccctgcactccgccatctcggcgggcactggagccagcggcattgtcgaggtcctcacggaggtgccaaacatcgatgttaccgccaccaacagccagggtttcaccctgctgcaccatgcctccctcaagggtcacgcgctagctgtgagaaagattctggctcgggcgcggcagctggtggacgccaagaaggaggacggcttcacggcgctgcatctggctgccctcaacaaccaccgcgaggtggcccagatcctcatccgggagggccgctgtgacgtgaacgtgcgcaaccggaagctgcagtccccgctgcatctcgccgtgcaacaggcccacgtggggctggtgccgctactggtggacgctgggtgcagtgtcaacgccgaggacgaggagggggacacagccctgcacgtggcgctgcagcgtcatcagctgctgcccctggtggctgatggggccgggggggacccagggcccttgcagctgctgtccaggctacaggcctcgggcctccccggcagcgcggagctgacggtgggcgcggcggtcgcctgcttcctggcgctggagggcgccgacgtgagctacaccaaccaccgcggtcggagcccgctggacctggccgccgagggtcgcgtgctcaaggcccttcagggctgcgcccagcgcttccgggagcggcaggcgggcgggggcgcggccccgggccccaggcaaacgctcgggacccccaacaccgtgacgaacctgcacgtgggcgccgcgccggggcccgaggccgctgagtgcctggtgtgctccgagctggcgctgctggtgctgttctcgccgtgccagcaccgcaccgtgtgtgaggagtgcgcgcgcaggatgaagaagtgcatcaggtgccaggtggtcgtcagcaagaaactgcgcccagacggctctgaggtggcgagcgccgcccccgcccccggcccgccgcgccagctggtggaggagctgcagagccgctaccggcagatggaggaacgcatcacctgccccatctgcatcgacagccacatccgcctcgtgttccagtgcggccacggcgcatgcgccccctgcggctccgcgctcagcgcctgccccatctgccgccagcccatccgcgaccgcatccagatcttcgtgtgagccgcgccgtccgccgcgcccgagctgccttcgcgtgcccccgccctgtgttttataaaaagaaagattctcgga

SEQ ID NO: 2 - protein, 599 aa, Homo sapiens
MAPALGRVGKVVKVFGDGNLRVAVAGQRWTFSPSCLVAYRPEEDANLDVAERARENKSSLSVALDKLRAQKSDPEHPGRLVVEVALGNAARALDLLRRRPEQVDTKNQGRTALQVAAYLGQVELIRLLLQARAGVDLPDDEGNTALHYAALGNQPEATRVLLSAGCRADAINSTQSTALHVAVQRGFLKVVRALCERGCDVNLPDAHSDTPLHSAISAGTGASGIVEVLTEVPNIDVTATNSQGFTLLHHASLKGHALAVRKILARARQLVDAKKEDGFTALHLAALNNHREVAQILIREGRCDVNVRNRKLQSPLHLAVQQAHVGLVPLLVDAGCSVNAEDEEGDTALHVALQRHQLLPLVADGAGGDPGPLQLLSRLQASGLPGSAELTVGAAVACFLALEGADVSYTNHRGRSPLDLAAEGRVLKALQGCAQRFRERQAGGGAAPGPRQTLGTPNTVTNLHVGAAPGPEAAECLVCSELALLVLFSPCQHRTVCEECARRMKKCIRCQVVVSKKLRPDGSEVASAAPAPGPPRQLVEELQSRYRQMEERITCPICIDSHIRLVFQCGHGACAPCGSALSACPICRQPIRDRIQIFV

SEQ ID NO: 3 - DNA, 1797 bp, Artificial Sequence
  Degenerate polynucleotide sequence of Zapop2
atggcnccngcnytnggnmgngtnggnaargtngtnaargtnttyggngayggnaayytnmgngtngcngtngcnggncarmgntggacnttywsnccnwsntgyytngtngcntaymgnccngargargaygcnaayytngaygtngcngarmgngcnmgngaraayaarwsnwsnytnwsngtngcnytngayaarytnmgngcncaraarwsngayccngarcayccnggnmgnytngtngtngargtngcnytnggnaaygcngcnmgngcnytngayytnytnmgnmgnmgnccngarcargtngayacnaaraaycarggnmgnacngcnytncargtngcngcntayytnggncargtngarytnathmgnytnytnytncargcnmgngcnggngtngayytnccngaygaygarggnaayacngcnytncaytaygcngcnytnggnaaycarccngargcnacnmgngtnytnytnwsngcnggntgymgngcngaygcnathaaywsnacncarwsnacngcnytncaygtngcngtncarmgnggnttyytnaargtngtnmgngcnytntgygarmgnggntgygaygtnaayytnccngaygcncaywsngayacnccnytncaywsngcnathwsngcnggnacnggngcnwsnggnathgtngargtnytnacngargtnccnaayathgaygtnacngcnacnaaywsncarggnttyacnytnytncaycaygcnwsnytnaarggncaygcnytngcngtnmgnaarathytngcnmgngcnmgncarytngtngaygcnaaraargargayggnttyacngcnytncayytngcngcnytnaayaaycaymgngargtngcncarathytnathmgngarggnmgntgygaygtnaaygtnmgnaaymgnaarytncarwsnccnytncayytngcngtncarcargcncaygtnggnytngtnccnytnytngtngaygcnggntgywsngtnaaygcngargaygargarggngayacngcnytncaygtngcnytncarmgncaycarytnytnccnytngtngcngayggngcnggnggngayccnggnccnytncarytnytnwsnmgnytncargcnwsnggnytnccnggnwsngcngarytnacngtnggngcngcngtngcntgyttyytngcnytngarggngcngaygtnwsntayacnaaycaymgnggnmgnwsnccnytngayytngcngcngarggnmgngtnytnaargcnytncarggntgygcncarmgnttymgngarmgncargcnggnggnggngcngcnccnggnccnmgncaracnytnggnacnccnaayacngtnacnaayytncaygtnggngcngcnccnggnccngargcngcngartgyytngtntgywsngarytngcnytnytngtnytnttywsnccntgycarcaymgnacngtntgygargartgygcnmgnmgnatgaaraartgyathmgntgycargtngtngtnwsnaaraarytnmgnccngayggnwsngargtngcnwsngcngcnccngcnccnggnccnccnmgncarytngtngargarytncarwsnmgntaymgncaratggargarmgnathacntgyccnathtgyathgaywsncayathmgnytngtnttycartgyggncayggngcntgygcnccntgyggnwsngcnytnwsngcntgyccnathtgymgncarccnathmgngaymgnathcarathttygtn

SEQ ID NO: 4 - protein, 8 aa, Artificial Sequence
  Flag peptide sequence
DYKDDDDK

SEQ ID NO: 5 - DNA, 20 bp, Artificial Sequence
  Oligonucleotide primer ZC15399
cgaccacctggcacctgatg

SEQ ID NO: 6 - DNA, 20 bp, Artificial Sequence
  Oligonucleotide primer ZC15400
cccaacaccgtgacgaacct

SEQ ID NO: 7 - DNA, 21 bp, Artificial Sequence
  Oligonucleotide primer ZC18622
tcctcgtcctcggcgttgaca

SEQ ID NO: 8 - DNA, 21 bp, Artificial Sequence
  Oligonucleotide primer ZC18623
tctggctgccctcaacaacca

SEQ ID NO: 9 - DNA, 25 bp, Artificial Sequence
  Oligonucleotide primer ZC14063
caccagacataatagctgacagact

SEQ ID NO: 10 - DNA, 18 bp, Artificial Sequence
  Oligonucleotide primer ZC18706
gggagggccgctgtgacg

SEQ ID NO: 11 - DNA, 18 bp, Artificial Sequence
  Oligonucleotide primer ZC18707
tcctcggcgtggacactg

(19) United States
(12) Patent Application Publication
(10) Pub. No.: US 2002/0042094 A1
(43) Pub. Date: Apr. 11, 2002

(54) RING finger protein zapop2

(76) Inventors: Domenick R. Venezia, Seattle, WA (US); David W. Taft, Seattle, WA (US); Theodore E. Whitmore, Redmond, WA (US)

Correspondence Address: Jennifer K. Johnson, J.D., ZymoGenetics, Inc., 1201 Eastlake Avenue East, Seattle, WA, 98102, US

(21) Appl. No.: 09/735,368

(22) Filed: Dec. 12, 2000

Related U.S. Application Data
(60) Provisional application No. 60/171,258, filed on Dec. 15, 1999.

(51) Int. Cl. C12Q 1/68; G01N 33/574; C12P 21/04; C07H 21/04; C12N 5/06; C12N 9/00

(52) U.S. Cl. 435/69.1; 435/70.21; 435/69.6; 435/325; 536/23.5; 435/6; 435/7.23; 435/183

(57) ABSTRACT

The present invention relates to polynucleotide and polypeptide molecules for zapop2, a novel human member of the RING finger protein group. The polypeptides, and polynucleotides encoding them, are expressed in specific human tissues, and may be used for detecting human chromosomal abnormalities at 1p36. The present invention also includes antibodies to the zapop2 polypeptides.

### REFERENCE TO RELATED APPLICATIONS

This application is related to Provisional Application 60/171,258, filed on Dec. 15, 1999. Under 35 U.S.C. §119(e)(1), this application claims benefit of said Provisional Application.

### BACKGROUND OF THE INVENTION

Proper control of the opposing processes of cell proliferation versus terminal differentiation and apoptotic programmed cell death is an important aspect of normal development and homeostasis (Raff, M. C., Cell 86:173-175, 1996), and has been found to be altered in many human diseases. See, for example, Sawyers, C. L. et al., Cell 64:337-350, 1991; Meyaard, L. et al., Science 257:217-219, 1992; Guo, Q. et al., Nature Med. 4:957-962, 1998; Barinaga, M., Science, 273:735-737, 1996; Solary, E. et al., Eur. Respir. J., 9:1293-1305, 1996; Hamet, P. et al., J. Hypertension 14:S65-S70, 1996; Roy, N. et al., Cell, 80:167-178, 1995; and Ambrosini, G., Nature Med., 8:917-921, 1997. Much progress has been made towards understanding the regulation of this balance. For example, signaling cascades have been elucidated through which extracellular stimuli, such as growth factors, peptide hormones, and cell-cell interactions, control the commitment of precursor cells to specific cell lineages and their subsequent proliferative expansion (Morrison, S. J. et al., Cell 88:287-298, 1997). Further, it has been found that cell cycle exit and terminal differentiation are coupled in most cell types. See, for example, Coppola, J. A. et al., Nature 320:760-763, 1986; Freytag, S. O., Mol. Cell. Biol. 8:1614-1624, 1988; Lee, E. Y. et al., Genes Dev. 8:2008-2021, 1994; Morgenbesser, S. D. et al., Nature 371:72-74, 1994; Casaccia-Bonnefil, P. et al., Genes Dev. 11:2335-2346, 1996; Zacksenhaus, E. et al., Genes Dev. 10:3051-3064, 1996; and Zhang, P. et al., Nature 387:151-158, 1997. Apoptosis also plays an important role in many developmental and homeostatic processes (Raff, M. C., Nature 356:397-400, 1992; Raff, M. C., supra.), and is often coordinately regulated with terminal differentiation (Jacobsen, K. A. et al., Blood 84:2784-2794, 1994; Morgenbesser et al., supra.; Yan, Y. et al., Genes Dev. 11:973-983, 1997; Zacksenhaus et al., supra.). Hence, it appears that the development of individual lineages, tissues, organs, or even entire multicellular organisms is the result of a finely tuned balance between increased cell production due to proliferation, and decreased numbers of cells resulting from terminal differentiation and apoptosis. This balance is most likely regulated coordinately by the convergence of multiple regulatory pathways. The identification of novel members of such networks can provide important insights into both normal cellular processes, as well as the etiology and treatment of human disease states.

Thus, there is a continuing need to discover new proteins that regulate proliferation, differentiation, and apoptotic pathways. The in vivo activities of inducers and inhibitors of these pathways illustrates

the enormous clinical potential of, and need for, novel proliferation, differentiation, and apoptotic proteins, their agonists and antagonists. The present invention addresses this need by providing such polypeptides for these and other uses that should be apparent to those skilled in the art from the teachings herein.

### BRIEF DESCRIPTION OF THE DRAWING

FIG. 1 is a hydrophobicity and secondary structure prediction plot of zapop2. The secondary structure prediction plot is based on Mehta, P. K. et al., Protein Science 4:2517-2525, 1995. The hydrophobicity plot is based on a sliding six-residue window, with buried G, S, and T residues and exposed H, Y, and W residues ignored; The hydrophobic ranking used was WFYILVMCAHTPGQNSRKDE (Trinquier, G., and Sanejuand, Y -H., Protein Engineer. 11:153-169, 1998). The first/right letter column in the plot depicts the amino acid. The plot also provides information on the domain structure (second/middle column) and secondary structure (third/left column) of the zapop2 polypeptide as follows: “*” in the second/middle column indicates the conserved Cysteine residues in the RING finger motifs; numbers 1 through 9 in the second/middle column indicate the nine ankyrin repeats; Helix character is indicated by “H” in the third/left column; Strand character is indicated by “E” in the third/left column; Coil character is indicated by “C” in the third/left column; Unpredicted structure is indicated by “X” in the third/left column.

### DESCRIPTION OF THE INVENTION

The present invention addresses this need by providing a novel polypeptide and related compositions and methods.

Within one aspect, the present invention provides an isolated polynucleotide that encodes a zapop2 polypeptide comprising a sequence of amino acid residues that is at least 90% identical to an amino acid sequence selected from the group consisting of: (a) the amino acid sequence as shown in SEQ ID NO:2 from amino acid number 476 (Cys), to amino acid number 599 (Val); and (b) the amino acid sequence as shown in SEQ ID NO:2 from amino acid number 1 (Met), to amino acid number 599 (Val), wherein the amino acid percent identity is determined using a FASTA program with ktup=1, gap opening penalty=10, gap extension penalty=1, and substitution matrix=BLOSUM62, with other parameters set as default. In one embodiment, the isolated polynucleotide disclosed above is selected from the group consisting of: (a) polynucleotide molecules comprising a nucleotide sequence as shown in SEQ ID NO:1 from nucleotide 1507 to nucleotide 1879; and (b) polynucleotide molecules comprising a nucleotide sequence as shown in SEQ ID NO: 1 from nucleotide 83 to nucleotide 1879; and (b) polynucleotide molecules complementary to (a). In one embodiment, the isolated polynucleotide disclosed above comprises nucleotide 1 to nucleotide 1797 of SEQ ID NO:3. In another embodiment, the isolated polynucleotide disclosed above comprises a polynucleotide encoding a polypeptide sequence of amino acid residues selected from the group consisting of: (a) the amino acid sequence as shown in SEQ ID NO:2 from amino acid number 476 (Cys), to amino acid number 599 (Val); and (b) the amino acid sequence as shown in SEQ ID NO:2 from amino acid number 1 (Met), to amino acid number 599 (Val). In another embodiment, the isolated polynucleotide disclosed above wherein the polynucleotide further encodes a polypeptide that contains at least one RING finger domain or at least one ankyrin repeat motif. In another embodiment, the isolated polynucleotide disclosed above, wherein the polynucleotide further encodes a polypeptide that contains at least one RING finger domain and at least one ankyrin repeat motif.

Within a second aspect, the present invention provides an expression vector comprising the following operably linked elements: a transcription promoter; a DNA segment encoding a zapop2 polypeptide having an amino acid sequence as shown in SEQ ID NO:2 from amino acid number 1 (Met), to amino acid number 599 (Val); and a transcription terminator, wherein the promoter is operably linked to the DNA segment, and the DNA segment is operably linked to the transcription terminator.

Within a third aspect, the present invention provides an expression vector as disclosed above, further comprising a secretory signal sequence operably linked to the DNA segment.

Within a fourth aspect, the present invention provides a cultured cell into which has been introduced an expression vector as disclosed above, wherein the cell expresses the polypeptide encoded by the DNA segment.

Within another aspect, the present invention provides a DNA construct encoding a fusion protein, the DNA construct comprising: a first DNA segment encoding a polypeptide comprising a sequence of amino acid residues selected from the group consisting of: (a) the amino acid sequence as shown in SEQ ID NO:2 from amino acid number 1 (Met), to amino acid number 74 (Pro); (b) the amino acid sequence as shown in SEQ ID NO:2 from amino acid number 75 (Glu), to amino acid number 376 (Leu);(c) the amino acid sequence as shown in SEQ ID NO:2 from amino acid number 476 (Cys), to amino acid number 510 (Cys); (d) the amino acid sequence as shown in SEQ ID NO:2 from amino acid number 555 (Cys), to amino acid number 587 (Cys); and at least one other DNA segment encoding an additional polypeptide,

wherein the first and other DNA segments are connected in-frame; and

wherein the first and other DNA segments encode the fusion protein.

Within another aspect, the present invention provides an expression vector comprising the following operably linked elements: a transcription promoter; a DNA construct encoding a fusion protein as disclosed above; and a transcription terminator, wherein the promoter is operably linked to the DNA construct, and the DNA construct is operably linked to the transcription terminator.

Within another aspect, the present invention provides a cultured cell comprising an expression vector as disclosed above, wherein the cell expresses a polypeptide encoded by the DNA construct.

Within another aspect, the present invention provides a method of producing a fusion protein comprising: culturing a cell as disclosed above; and isolating the polypeptide produced by the cell.

Within another aspect, the present invention provides an isolated polypeptide comprising a sequence of amino acid residues that is at least 90% identical to an amino acid sequence selected from the group consisting of: (a) the amino acid sequence as shown in SEQ ID NO:2 from amino acid number 476 (Cys), to amino acid number 599 (Val); and (b) the amino acid sequence as shown in SEQ ID NO:2 from amino acid number 1 (Met), to amino acid number 599 (Val), wherein the amino acid percent identity is determined using a FASTA program with ktup=1, gap opening penalty=10, gap extension penalty=1, and substitution matrix=BLOSUM62, with other parameters set as default. In one embodiment, the isolated polypeptide disclosed above comprises a sequence of amino acid residues selected from the group consisting of: (a) the amino acid sequence as shown in SEQ ID NO:2 from amino acid number 476 (Cys), to amino acid number 599 (Val); and (b) the amino acid sequence as shown in SEQ ID NO:2 from amino acid number 1 (Met), to amino acid number 599 (Val). In another embodiment, the isolated polypeptide disclosed above further contains at least one RING finger domain or at least one ankyrin repeat motif. In another embodiment, the isolated polypeptide disclosed above further contains at least one RING finger domain and at least one ankyrin repeat motif.

Within another aspect, the present invention provides a method of producing a zapop2 polypeptide comprising: culturing a cell as disclosed above; and isolating the zapop2 polypeptide produced by the cell.

Within another aspect, the present invention provides a method of producing an antibody to zapop2 polypeptide comprising: inoculating an animal with a polypeptide selected from the group consisting of: (a) a polypeptide consisting of 9 to 599 amino acids, wherein the polypeptide consists of a contiguous sequence of amino acids in SEQ ID NO:2 from amino acid number 1 (Met), to amino acid number 599 (Val); (b) a polypeptide as disclosed above; (c) a polypeptide consisting of the amino acid sequence of SEQ ID NO:2 from amino acid residue 51 (Glu) to amino acid number 56 (Asn); (d) a polypeptide consisting of the amino acid sequence of SEQ ID NO:2 from amino acid residue 272 (Asp) to amino acid number 277 (Asp); (e) a polypeptide consisting of the amino acid sequence of SEQ ID NO:2 from amino acid residues 339 (Asn) to amino acid number 344 (Glu); (f) a polypeptide consisting of the amino acid sequence of SEQ ID NO:2 from amino acid number 341 (Glu) to amino acid number 346 (Asp), wherein the polypeptide elicits an immune response in the animal to produce the antibody; and isolating the antibody from the animal.

Within another aspect, the present invention provides an antibody produced by the method as disclosed above, which binds to a zapop2 polypeptide. In another embodiment, the antibody disclosed above is a monoclonal antibody. Within another aspect, the present invention provides an antibody which binds to a polypeptide as disclosed above.

Within another aspect, the present invention provides a method of detecting, in a test sample, the presence of an agonist of zapop2 protein activity, comprising: transfecting a zapop2-expressing cell, with a reporter gene construct that is responsive to a zapop2-stimulated cellular pathway; and adding a test sample; and comparing levels of response in the presence and absence of the test sample, by a biological or biochemical assay; and determining from the comparison, the presence of the agonist of zapop2 activity in the test sample.

Within another aspect, the present invention provides a method for detecting a genetic abnormality in a patient, comprising: obtaining a genetic sample from a patient; producing a first reaction product by incubating the genetic sample with a polynucleotide comprising at least 14 contiguous nucleotides of SEQ ID NO: 1 or the complement of SEQ ID NO: 1, under conditions wherein said polynucleotide will hybridize to complementary polynucleotide sequence; visualizing the first reaction product; and comparing said first reaction product to a control reaction product from a wild type patient, wherein a difference between said first reaction product and said control reaction product is indicative of a genetic abnormality in the patient.

Within another aspect, the present invention provides a method for detecting a cancer in a patient, comprising: obtaining a tissue or biological sample from a patient; incubating the tissue or biological sample with an antibody as disclosed above under conditions wherein the antibody binds to its complementary polypeptide in the tissue or biological sample; visualizing the antibody bound in the tissue or biological sample; and comparing levels of antibody bound in the tissue or biological sample from the patient to a normal control tissue or biological sample, wherein an increase or decrease in the level of antibody bound to the patient tissue or biological sample relative to the normal control tissue or biological sample is indicative of a cancer in the patient.

Within another aspect, the present invention provides a method for detecting a cancer in a patient, comprising: obtaining a tissue or biological sample from a patient; labeling a polynucleotide comprising at least 14 contiguous nucleotides of SEQ ID NO: 1 or the complement of SEQ ID NO: 1; incubating the tissue or biological sample with under conditions wherein the polynucleotide will hybridize to complementary polynucleotide sequence; visualizing the labeled polynucleotide in the tissue or biological sample; and comparing the level of labeled polynucleotide hybridization in the tissue or biological sample from the patient to a normal control tissue or biological sample, wherein an increase or decrease in the labeled polynucleotide hybridization to the patient tissue or biological sample relative to the normal control tissue or biological sample is indicative of a cancer in the patient.

These and other aspects of the invention will become evident upon reference to the following detailed description of the invention.

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzmol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complement/anti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” denotes a polynucleotide molecule having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGAGCTT-3′ are 5′-AGCTTgagt-3′ and 3′-tcgacTACC-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation. “Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “polypeptide” is a polymer of amino acid residues joined by peptide bonds, whether produced naturally or synthetically. Polypeptides of less than about 10 amino acid residues are commonly referred to as “peptides”.

“Probes and/or primers” as used herein can be RNA or DNA. DNA can be either cDNA or genomic DNA. Polynucleotide probes and primers are single or double-stranded DNA or RNA, generally synthetic oligonucleotides, but may be generated from cloned cDNA or genomic sequences or its complements. Analytical probes will generally be at least 20 nucleotides in length, although somewhat shorter probes (14-17 nucleotides) can be used. PCR primers are at least 5 nucleotides in length, preferably 15 or more nt, more preferably 20-30 nt. Short polynucleotides can be used when a small region of the gene is targeted for analysis. For gross analysis of genes, a polynucleotide probe may comprise an entire exon or more. Probes can be labeled to provide a detectable signal, such as with an enzyme, biotin, a radionuclide, fluorophore, chemiluminescer, paramagnetic particle and the like, which are commercially available from many sources, such as Molecular Probes, Inc., Eugene, Oreg., and Amersham Corp., Arlington Heights, Ill., using techniques that are well known in the art.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an mRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that encodes a polypeptide having partial homology to the BRCA1 RING finger domain (Jensen, D. E. et al., Oncogene 16:1097-1112, 1998) and containing ankyrin repeats. Analysis of the tissue distribution of the mRNA corresponding to this novel DNA showed strong expression levels in heart and skeletal muscle, and low expression in other tissues. The polypeptide has been designated zapop2.

The novel zapop2 polypeptides of the present invention were initially identified by querying an EST database for proteins homologous to proteins having a RING finger sequence. The consensus RING finger motifs are characterized by cysteine motifs of the formula:

CXXCX{11-12}CXHX{3}CXXCX{6-11}CXXC,

or

CXXCX{10-27}CXHX{2-3}CXXCX{5-16}CPXC,

wherein

X is any amino acid,

C is Cysteine,

H is Histidine, and

{#-#} is the range of repetition of the preceding residue X.

These cysteine motifs occur in all currently known RING finger proteins, such as, apoptosis inhibitor proteins (IAPs), and the like, and is unique to this group of proteins. These search criteria were compared to an EST database to identify novel proteins having homology to known RING finger proteins. The full sequence of the zapop2 polypeptide was obtained from a single clone believed to contain it, wherein the clone was obtained from a peripheral blood granulocyte library. Other libraries that might also be searched for such sequences include heart, skeletal muscle, pancreas, brain, stomach, colon, thyroid, and the like.

The nucleotide sequence of full-length zapop2 is described in SEQ ID NO:1, and its deduced amino acid sequence is described in SEQ ID NO:2. Sequence analysis revealed that zapop2 is a member of a diverse group of proteins that contain ankyrin repeats, and is a member of a diverse group of proteins that are characterized by a RING finger domain. Zapop2, unlike known ankyrin repeat or RING finger proteins, contains both the RING finger domain and ankyrin repeats in the same protein.

Analysis of the DNA encoding zapop2 polypeptide (SEQ ID NO:1) revealed an open reading frame encoding 599 amino acids (SEQ ID NO:2), encoding an approximately 64 kD protein. Multiple alignment of zapop2 with BRCA1, and other members of the RING finger protein group, such as murine and human IAPs in addition to structural determinations based on amino acid sequences revealed the following regions, domains, and conserved motifs:

(1) An N-terminal domain corresponding to amino acid residues 1 (Met) to amino acid residue 74 (Pro) of SEQ ID NO:2;

(2) An ankyrin repeat region, corresponding to amino acid residues 75 (Glu) to amino acid residue 376 (Leu) of SEQ ID NO:2. Within the ankyrin repeat region there are 9 consecutive ankyrin repeat motifs, ordered from N-terminus to C-terminus: “ankyrin repeat-1” (corresponding to amino acids 75 (Glu) to 107 (Asn) of SEQ ID NO:2); “ankyrin repeat-2” (corresponding to amino acids 108 (Gln) to 140 (Asp) of SEQ ID NO:2); “ankyrin repeat-3” (corresponding to amino acids 141 (Glu) to 173 (Ser) of SEQ ID NO:2); “ankyrin repeat-4” (corresponding to amino acids 174 (Thr) to 206 (Ala) of SEQ ID NO:2); “ankyrin repeat-5” (corresponding to amino acids 207 (His) to 242 (Ser) of SEQ ID NO:2); “ankyrin repeat-6” (corresponding to amino acids 243 (Gln) to 276 (Glu) of SEQ ID NO:2); “ankyrin repeat-7” (corresponding to amino acids 277 (Asp) to 310 (Arg) of SEQ ID NO:2); “ankyrin repeat-8” (corresponding to amino acids 311 (Lys) to 343 (Glu) of SEQ ID NO:2); and “ankyrin repeat-9” (corresponding to amino acids 344 (Glu) to 376 (Leu) of SEQ ID NO:2);

(3) A Central region, corresponding to amino acid residues 377 (Ser) to amino acid residue 475 (Glu) of SEQ ID NO:2; and

(4) A C-terminal region, corresponding to amino acid residues 476 (Cys) to amino acid residue 599 (Val) of SEQ ID NO:2. Within this region are two RING finger domains: RING finger domain-1 (RING-1) corresponding to amino acid residues 476 (Cys) to amino acid residue 510 (Cys) of SEQ ID NO:2; and RING finger domain-2 (RING-2) corresponding to amino acid residues 555 (Cys) to amino acid residue 587 (Cys) of SEQ ID NO:2. RING finger domain-1 contains the first RING finger consensus sequence described above; and RING finger domain-2 contains the second RING finger consensus sequence described above.

The presence of conserved and low variance motifs generally correlates with or defines important structural regions in proteins. Regions of low variance (e.g., hydrophobic clusters) are generally present in regions of structural importance (Sheppard, P. et al., Gene 150:163-167, 1994). Such regions of low variance often contain rare or infrequent amino acids, such as Tryptophan. The regions flanking and between such conserved and low variance motifs may be more variable, but are often functionally significant because they may relate to or define important structures and activities such as binding domains, biological and enzymatic activity, signal transduction, tissue localization domains and the like. For example, ankyrin repeats are regions of protein-protein interaction and as such the ankyrin domain may be responsible for effecting signal transduction, homodimerization or heterodimerization of zapop2. Moreover, ankyrin repeats are involved with interactions with the cytoskeleton, focal adhesions, and spectrin binding. Moreover, the N- and C-terminal regions, described above may be functionally significant. Moreover, some domains, such as the RING finger domain and ankyrin repeat motifs, have known biological activities, for example as protein binding or DNA binding domains (Wang, H. et al., Oncogene 15:143-157, 1997; Buchanan, S. and Gay, N., Prog. Biophys. Molec. Biol. 65:1-44, 1996; Brzovic, P. S. et al., J. Biol. Chem. 273:7795-7799, 1998). The corresponding polynucleotides encoding the zapop2 polypeptide regions, domains, motifs and residues and sequences described above are as shown in SEQ ID NO:1.

The conserved amino acids in the ankyrin repeat region and the RING finger domain of zapop2 can be used as a tool to identify new group members. For instance, reverse transcription-polymerase chain reaction (RT-PCR) can be used to amplify sequences encoding the conserved RING finger motif from RNA obtained from a variety of tissue sources or cell lines. In particular, highly degenerate primers designed from the zapop2 polynucleotide sequences are useful for this purpose. Designing and using such degenerate primers may be readily performed by one skilled in the art. The present invention also provides polynucleotide molecules, including DNA and RNA molecules, that encode the zapop2 polypeptides disclosed herein. Those skilled in the art will readily recognize that, in view of the degeneracy of the genetic code, considerable sequence variation is possible among these polynucleotide molecules. SEQ ID NO:3 is a degenerate DNA sequence that encompasses all DNAs that encode the zapop2 polypeptide of SEQ ID NO:2. Those skilled in the art will recognize that the degenerate sequence of SEQ ID NO:3 also provides all RNA sequences encoding SEQ ID NO:2 by substituting U for T. Thus, zapop2 polypeptide-encoding polynucleotides comprising nucleotide 1 to nucleotide 1797 of SEQ ID NO:3 and their RNA equivalents are contemplated by the present invention. Table 1 sets forth the one-letter codes used within SEQ ID NO:3 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:3, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. Biol. 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid Threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:3 serves as a template for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention the isolated polynucleotides will hybridize to similar sized regions of SEQ ID NO:1, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227, 1990). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences (e.g., >50 base pairs) is performed at temperatures of about 20-25° C. below the calculated Tₘ. For smaller probes (e.g., <50 base pairs) hybridization is typically carried out at the Tₘ or 5-10° C. below. This allows for the maximum rate of hybridization for DNA-DNA and DNA-RNA hybrids. Higher degrees of stringency at lower temperatures can be achieved with the addition of formamide which reduces the Tₘ of the hybrid about 1° C. for each 1% formamide in the buffer solution. Suitable stringent hybridization conditions are equivalent to about a 5 h to overnight incubation at about 42° C. in a solution comprising: about 40-50% formnamide, up to about 6×SSC, about 5×Denhardt's solution, zero up to about 10% dextran sulfate, and about 10-20 μg/ml denatured commercially-available carrier DNA. Generally, such stringent conditions include temperatures of 20-70° C. and a hybridization buffer containing up to 6×SSC and 0-50% formamide; hybridization is then followed by washing filters in up to about 2×SSC. For example, a suitable wash stringency is equivalent to 0.1×SSC to 2×SSC, 0.1% SDS, at 55° C. to 65° C. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes. Stringent hybridization and wash conditions depend on the length of the probe, reflected in the Tm, hybridization and wash solutions used, and are routinely determined empirically by one of skill in the art.

As previously noted, the isolated polynucleotides of the present invention include DNA and RNA. Methods for preparing DNA and RNA are well known in the art. In general, RNA is isolated from a tissue or cell that produces large amounts of zapop2 RNA. Such tissues and cells are identified by Northern blotting (Thomas, Proc. Natl. Acad. Sci. USA 77:5201, 1980), and include heart and skeletal muscle. Total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding zapop2 polypeptides are then identified and isolated by, for example, hybridization or polymerase chain reaction (PCR) (Mullis, U.S. Pat. No. 4,683,202).

A full-length clone encoding zapop2 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zapop2, polypeptide fragments thereof, or other specific binding partners.

The polynucleotides of the present invention can also be synthesized using DNA synthesis machines, for example, by using the phosphoramidite method. A synthetic zapop2 gene can be constructed from a set of overlapping, complementary oligonucleotides, each of which is between 20 to 60 nucleotides long. Each internal section of the gene has complementary 3′ and 5′ terminal extensions designed to base pair precisely with an adjacent section. Thus, after the gene is assembled, process is completed by sealing the nicks along the backbones of the two strands with T4 DNA ligase. In addition to the protein coding sequence, synthetic genes can be designed with terminal sequences that facilitate insertion into a restriction endonuclease site of a cloning vector. Moreover, other sequences should can be added that contain signals for proper initiation and termination of transcription and translation. Alternatively, a specified set of partially overlapping oligonucleotides (40 to 100 nucleotides) can be used. After the 3′ and 5′ short overlapping complementary regions are paired, large gaps may still remain, but the base-paired regions are both long enough and stable enough to hold the structure together. The gaps are filled, and the DNA duplex is completed, via enzymatic DNA synthesis by E. coli DNA polymerase I. After the enzymatic synthesis is completed, the nicks are sealed with T4 DNA ligase. Double-stranded constructs are sequentially linked to one another to form the entire gene sequence which is verified by DNA sequence analysis. See Glick and Pasternak, Molecular Biotechnology, Principles & Applications of Recombinant DNA, (ASM Press, Washington, D.C. 1994); Itakura et al., Annu. Rev. Biochem. 53: 323-56, 1984 and Climie et al., Proc. Natl. Acad. Sci. USA 87:633-7, 1990.

Zapop2 polynucleotide sequences disclosed herein can also be used as probes or primers to clone 5′ non-coding regions of a zapop2 gene. In view of the tissue-specific expression observed for zapop2 by Northern blotting, this gene region is expected to provide for heart- and skeletal muscle-specific expression. Promoter elements from a zapop2 gene could thus be used to direct the tissue-specific expression of heterologous genes in, for example, transgenic animals or patients treated with gene therapy. Cloning of 5′ flanking sequences also facilitates production of zapop2 proteins by “gene activation” as disclosed in U.S. Pat. No. 5,641,670. Briefly, expression of an endogenous zapop2 gene in a cell is altered by introducing into the zapop2 locus a DNA construct comprising at least a targeting sequence, a regulatory sequence, an exon, and an unpaired splice donor site. The targeting sequence is a zapop2 5′ non-coding sequence that permits homologous recombination of the construct with the endogenous zapop2 locus, whereby the sequences within the construct become operably linked with the endogenous zapop2 coding sequence. In this way, an endogenous zapop2 promoter can be replaced or supplemented with other regulatory sequences to provide enhanced, tissue-specific, or otherwise regulated expression.

The present invention further provides counterpart polypeptides and polynucleotides from other species (orthologs). These species include, but are not limited to mammalian, avian, amphibian, reptile, fish, insect and other vertebrate and invertebrate species. Of particular interest are zapop2 polypeptides from other mammalian species, including murine, porcine, ovine, bovine, canine, feline, equine, and other primate polypeptides. Orthologs of human zapop2 can be cloned using information and compositions provided by the present invention in combination with conventional cloning techniques. For example, a cDNA can be cloned using mRNA obtained from a tissue or cell type that expresses zapop2 as disclosed herein. Suitable sources of mRNA can be identified by probing Northern blots with probes designed from the sequences disclosed herein. A library is then prepared from mRNA of a positive tissue or cell line. A zapop2-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human zapop2sequence disclosed herein. Within an additional method, the cDNA library can be used to transform or transfect host cells, and expression of the cDNA of interest can be detected with an antibody to zapop2 polypeptide. Similar techniques can also be applied to the isolation of genomic clones.

Those skilled in the art will recognize that the sequence disclosed in SEQ ID NO:1 represents a single allele of human zapop2 and that allelic variation and alternative splicing are expected to occur. Allelic variants of this sequence can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the zapop2 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art. The corresponding polynucleotides encoding the zapop2 polypeptide regions, domains, motifs, residues and sequences described above are as shown in SEQ ID NO:1.

The present invention also provides isolated zapop2 polypeptides that are substantially similar to the polypeptides of SEQ ID NO:2 and their orthologs. The term “substantially similar” is used herein to denote polypeptides having 50%, preferably 60%, more preferably at least 80%, sequence identity to the sequences shown in SEQ ID NO:2 or their orthologs. Such polypeptides will more preferably be at least 90% identical, and most preferably 95% or more identical to SEQ ID NO:2 or its orthologs.) Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 3 (amino acids are indicated by the standard one-letter codes). The percent identity is then calculated as:

$$\frac{Total number of identical matches}{\text{[length of the longer sequence plus the number of gaps introduced into the longer sequence in order to align the two sequences]}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zapop2. The FASTA algorithm is described by Pearson and Lipman, Proc. Nat'l Acad. Sci. USA 85:2444, 1988; and by Pearson, Meth. Enzymol. 183:63, 1990.

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol., supra.

FASTA can also be used to determine the sequence identity of nucleic acid molecules using a ratio as disclosed above. For nucleotide sequence comparisons, the ktup value can range between one to six, preferably from three to six, most preferably three, with other FASTA program parameters set as default.

The BLOSUM62 table (Table 3) is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915, 1992). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed below), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Variant zapop2 polypeptides or substantially homologous zapop2 polypeptides are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and small amino- or carboxyl-terminal extensions, such as an amino-terminal methionine residue, a linker peptide of up to about 20-25 residues, or an affinity tag. The present invention thus includes polypeptides of from about 475 to about 625 amino acid residues that comprise a sequence that is at least 80%, preferably at least 90%, and more preferably 95% or more identical to the corresponding region of SEQ ID NO:2. Polypeptides comprising affinity tags can further comprise a proteolytic cleavage site between the zapop2 polypeptide and the affinity tag. Preferred such sites include thrombin cleavage sites and factor Xa cleavage sites.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The present invention further provides a variety of other polypeptide fusions and related multimeric proteins comprising one or more polypeptide fusions. For example, a zapop2 polypeptide can be prepared as a fusion to a dimerizing protein as disclosed in U.S. Pat. Nos. 5,155,027 and 5,567,584. Preferred dimerizing proteins in this regard include immunoglobulin constant region domains. Immunoglobulin-zapop2 polypeptide fusions can be expressed in genetically engineered cells to produce a variety of multimeric zapop2 analogs. Auxiliary domains can be fused to zapop2 polypeptides to target them to specific cells, tissues, or macromolecules (e.g., collagen). For example, a zapop2 polypeptide or protein could be targeted to a predetermined cell type by fusing a zapop2 polypeptide to a ligand that specifically binds to a receptor on the surface of the target cell. In this way, polypeptides and proteins can be targeted for therapeutic or diagnostic purposes. A zapop2 polypeptide can be fused to two or more moieties, such as an affinity tag for purification and a targeting domain. Polypeptide fusions can also comprise one or more cleavage sites, particularly between domains. See, Tuan et al., Connective Tissue Research 34:1-9, 1996.

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for zapop2 amino acid residues.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of ligand-receptor or other biological or functional interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related ankyrin repeat-containing and RING finger proteins.

Determination of amino acid residues that are within regions or domains that are critical to maintaining structural integrity can be determined. Within these regions one can determine specific residues that will be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in zapop2 polypeptides so as to minimize disruption of higher order structure essential to biological activity. For example, when the zapop2 polypeptide comprises one or more structural domains, such as RING domains, changes in amino acid residues will be made so as not to disrupt the structure geometry and other components of the molecule where changes in conformation abate some critical function, for example, binding of the molecule to its binding partners. The effects of amino acid sequence changes can be predicted by, for example, computer modeling as disclosed above or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same disulfide bonding pattern as the standard molecule folding would be affected. Another well known and accepted method for measuring folding is circular dichroism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structural similarities between proteins and polypeptides (Schaanan et al., Science 257:961-964, 1992).

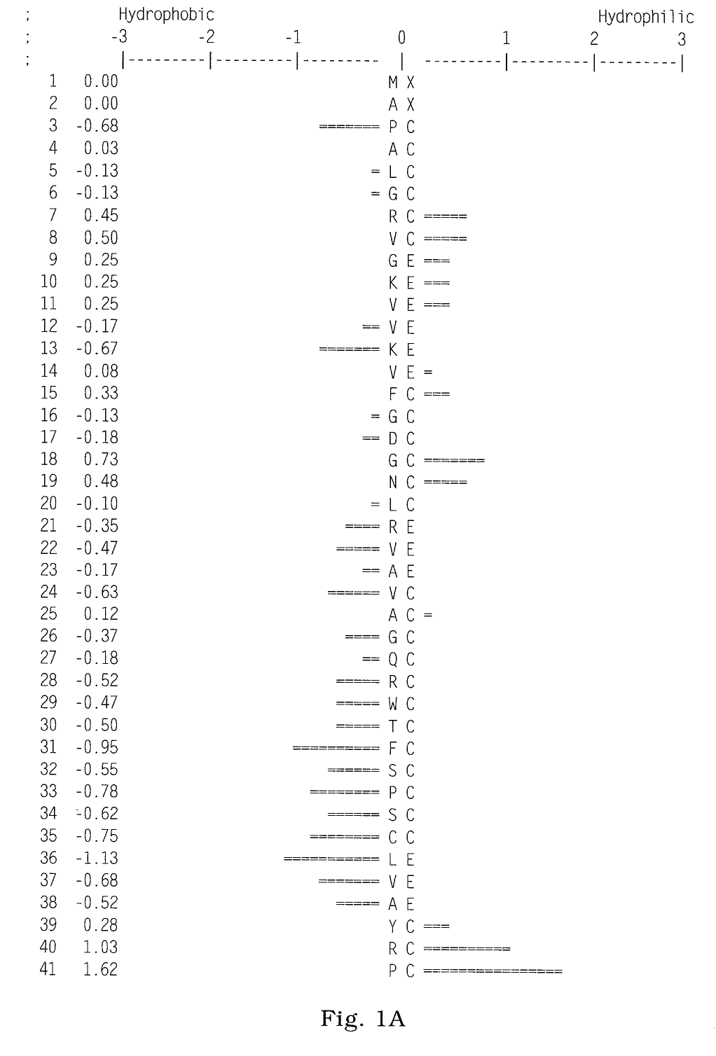

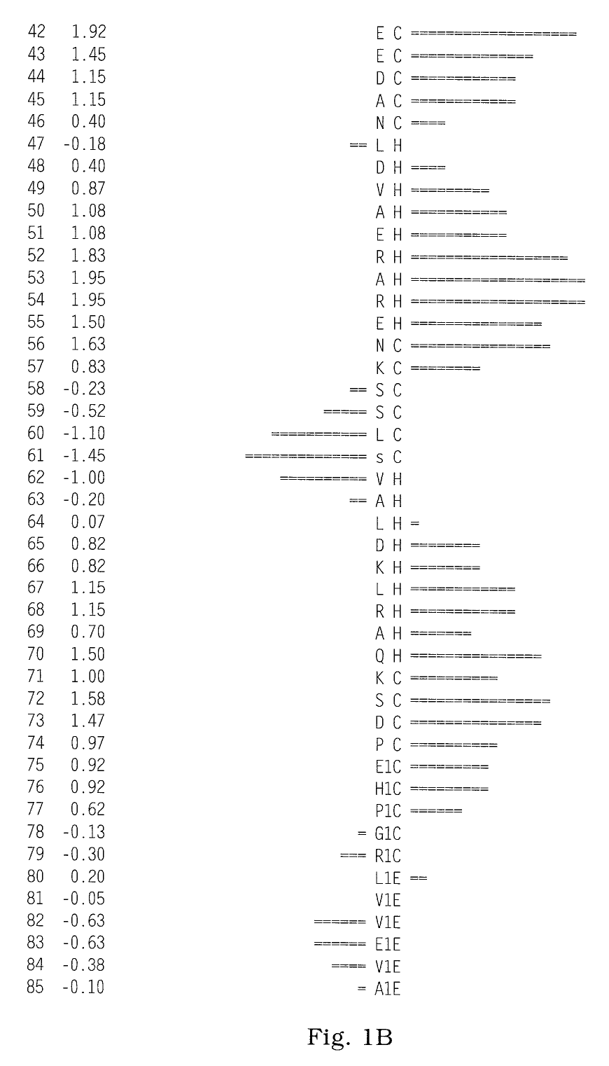

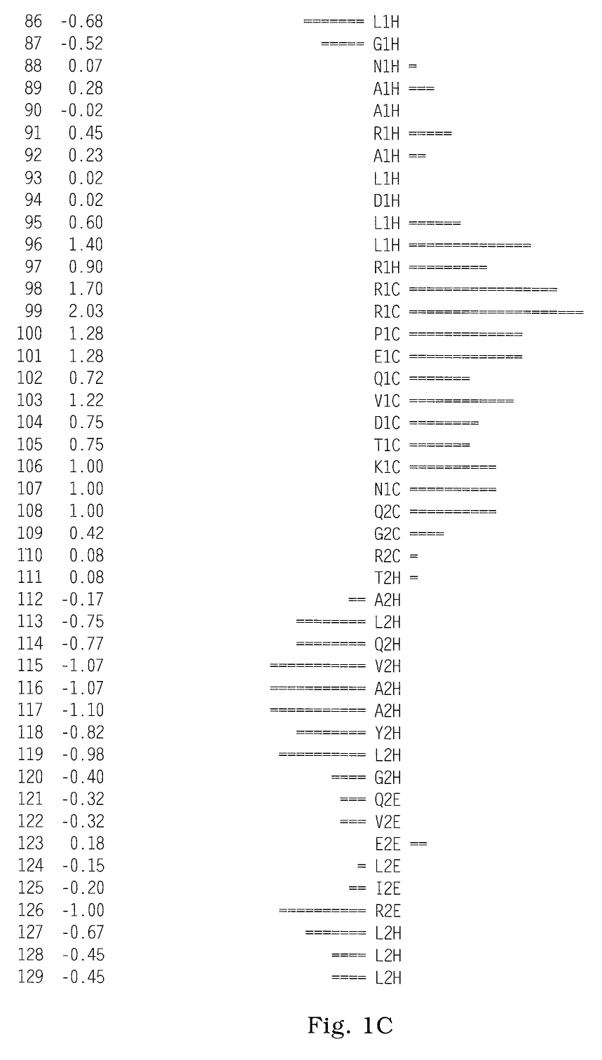

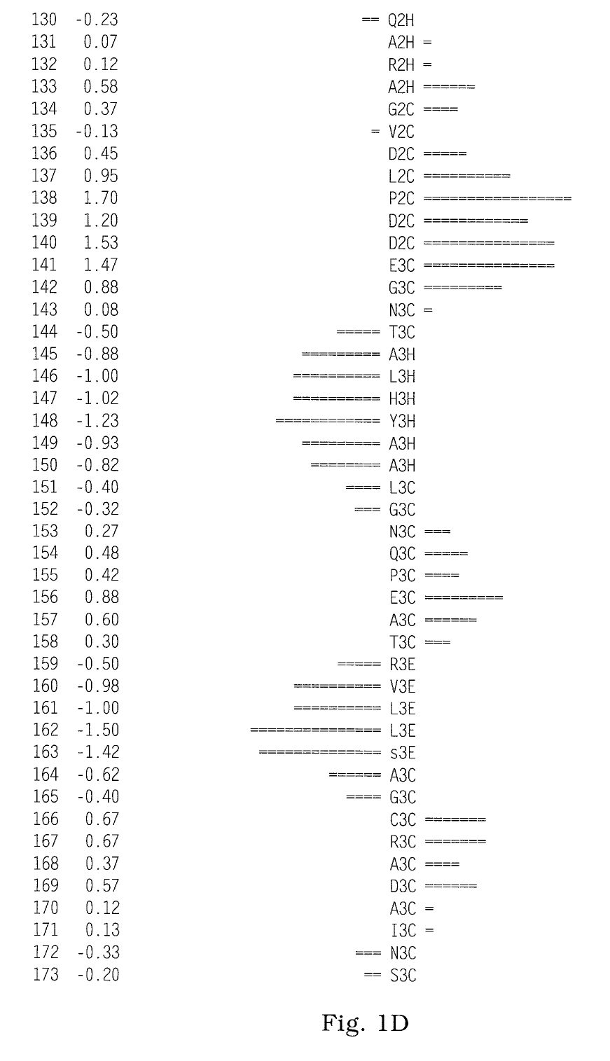

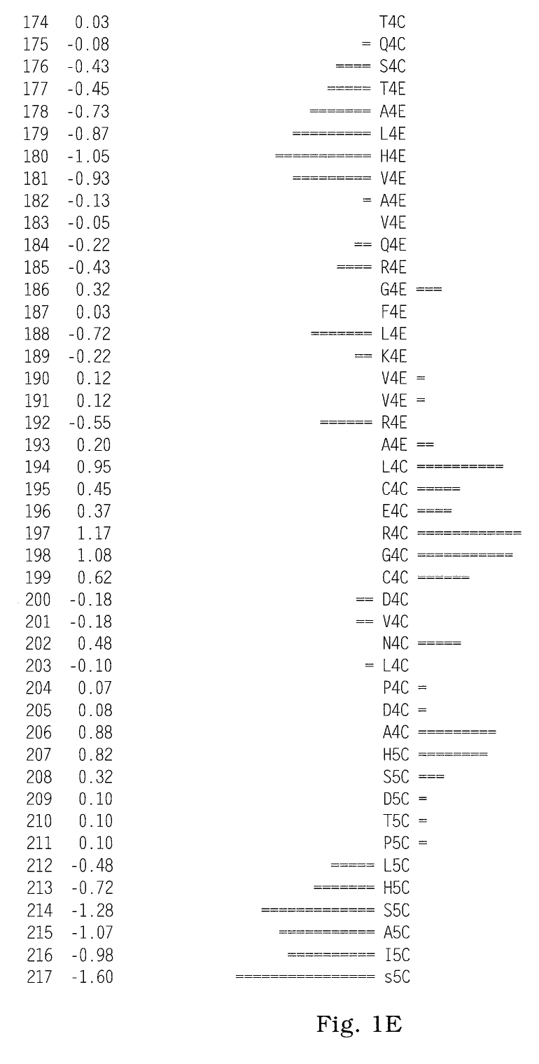

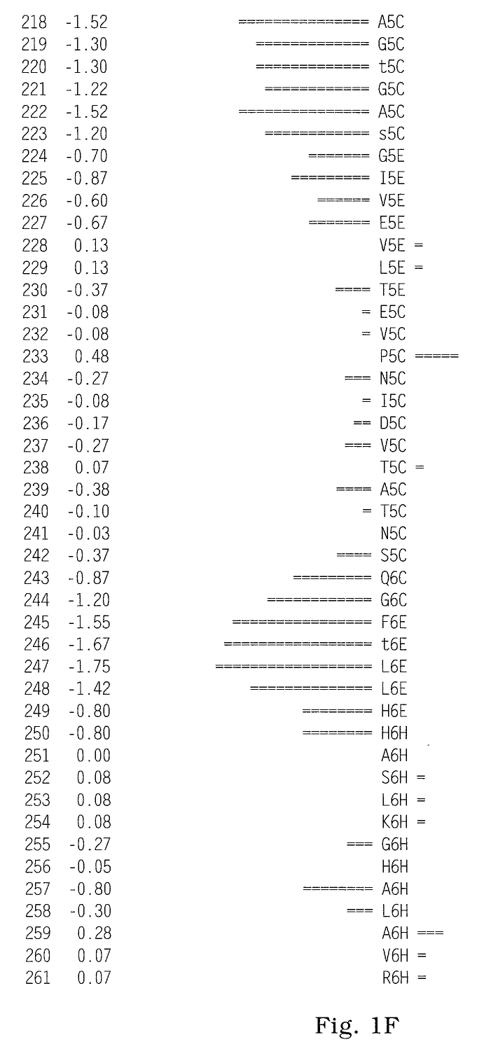

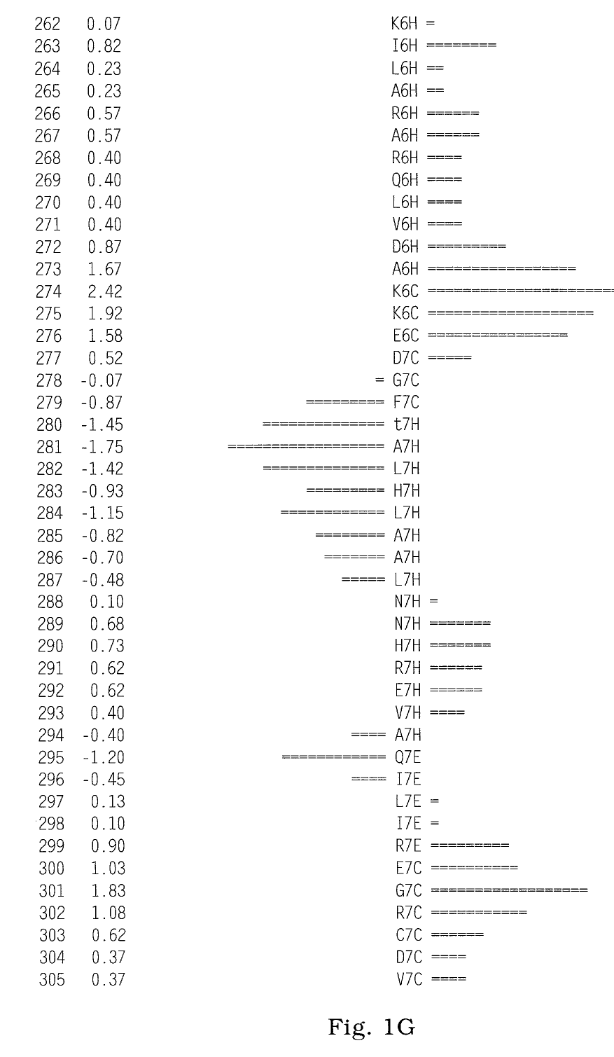

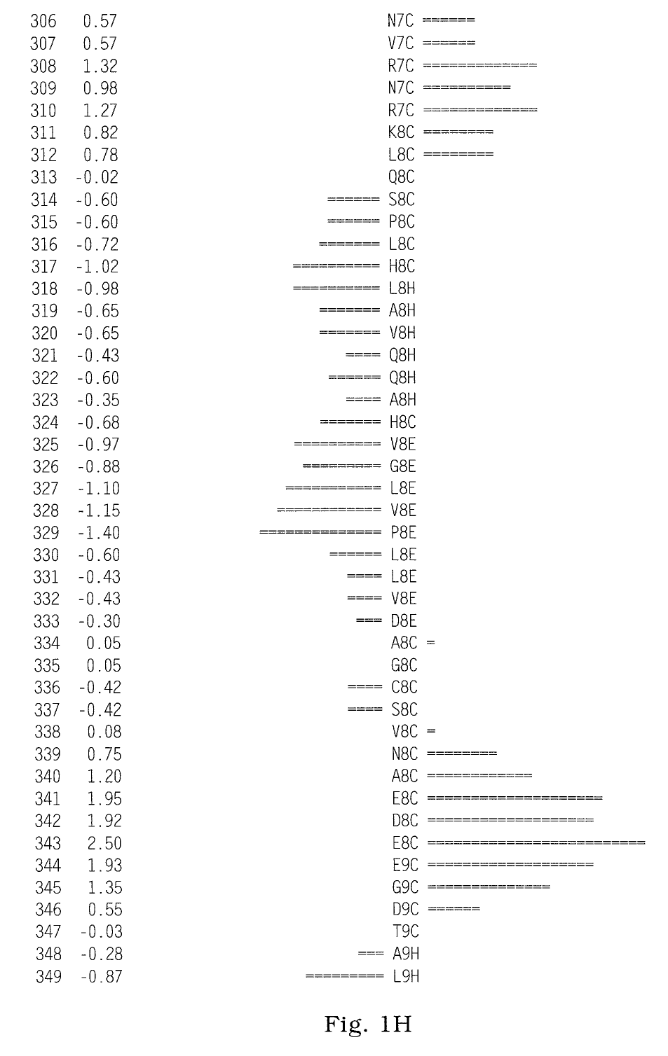

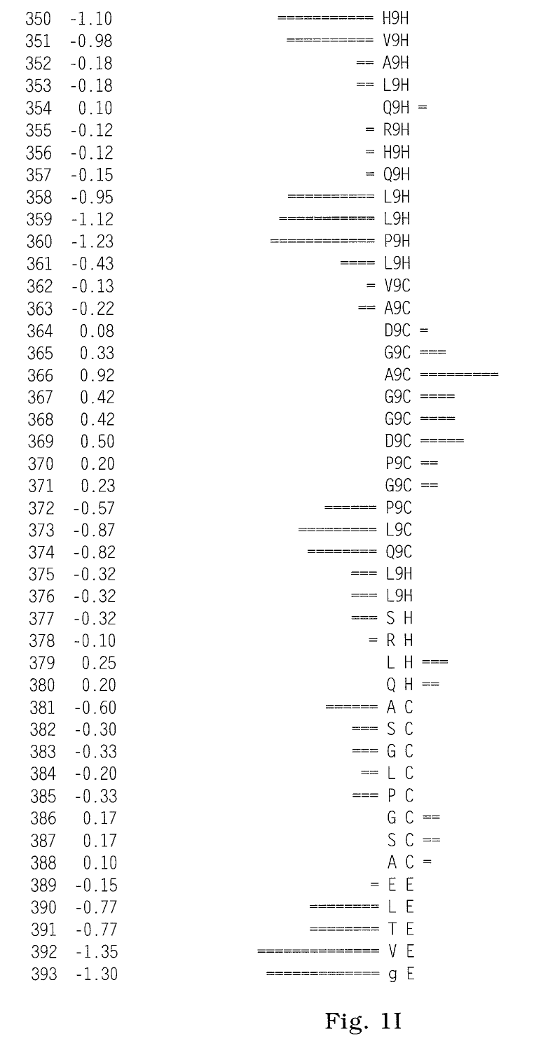

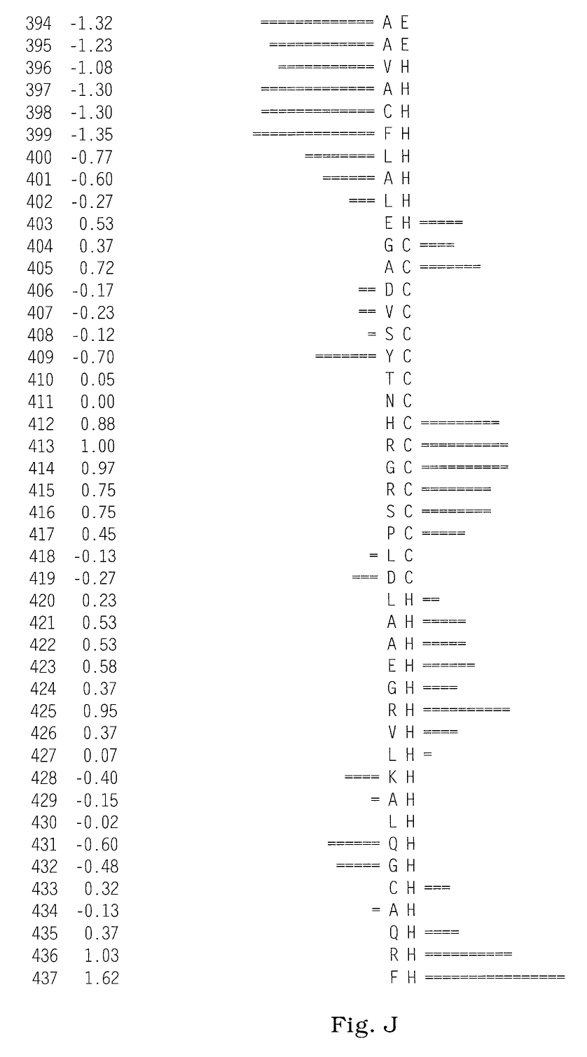

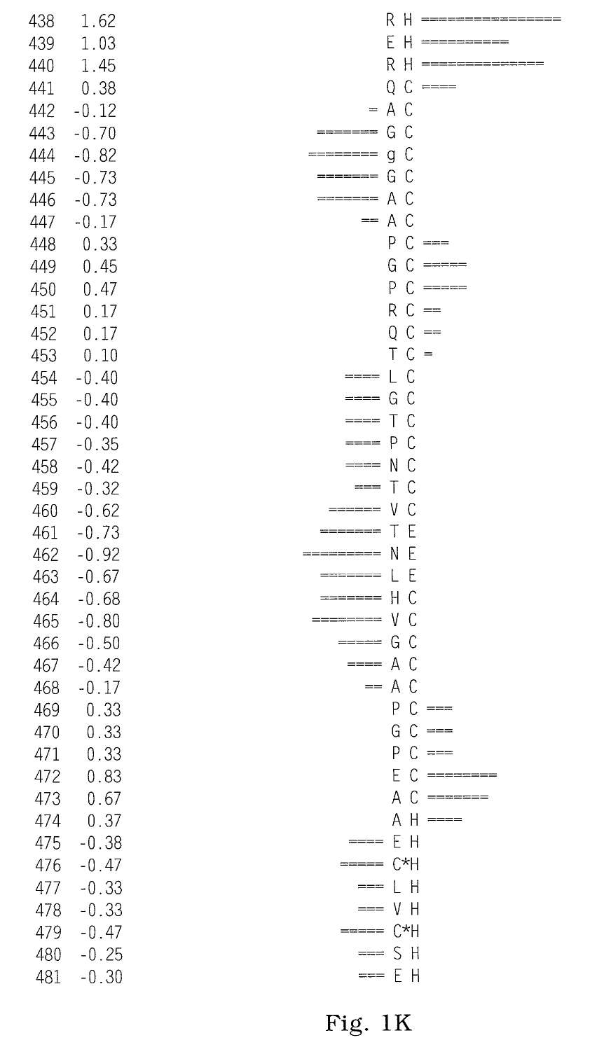

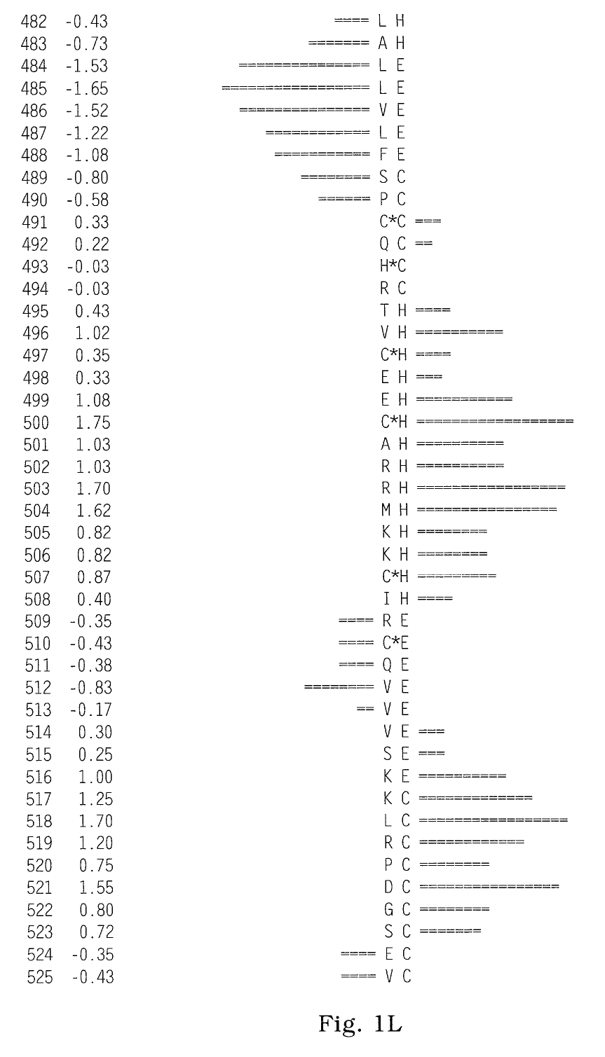

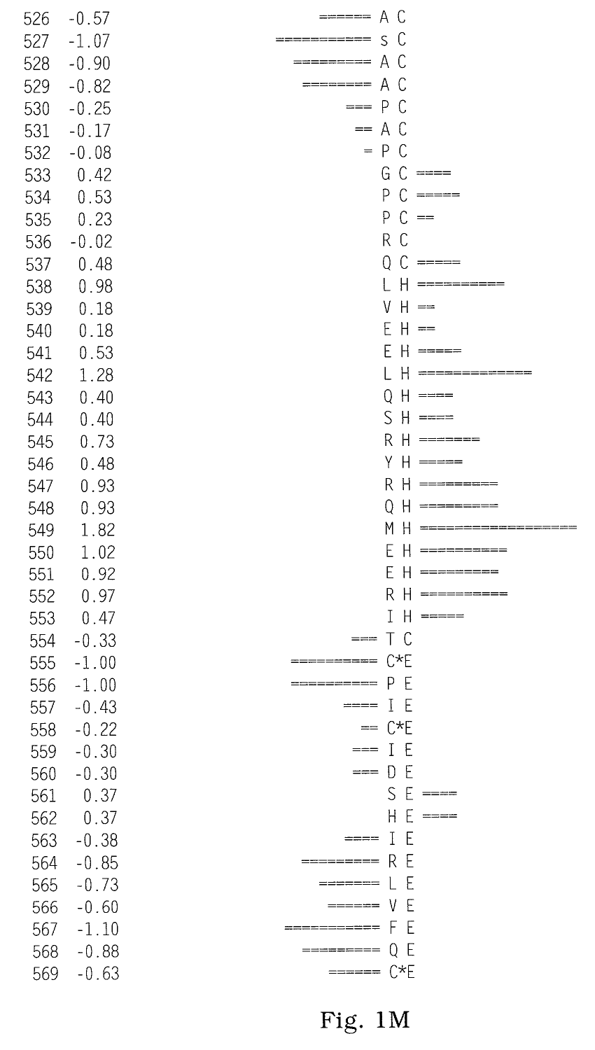

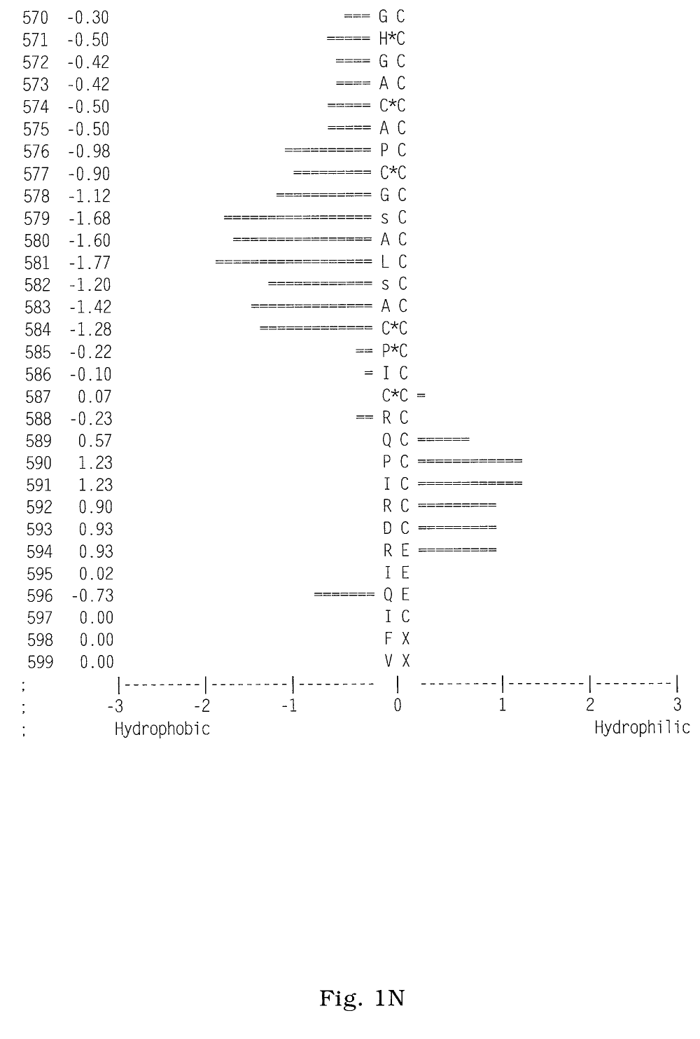

A hydrophilicity profile of the zapop2 protein sequence as shown in SEQ ID NO:2 can be generated (Hopp et al., Proc. Natl. Acad. Sci. 78:3824-3828, 1981; Hopp, J. Immun. Meth. 88:1-18, 1986 and Triquier et al., Protein Engineering 11:153-169, 1998). For example, see FIG. 1. For example, in zapop2, hydrophilic regions include: (1) amino acid number 51 (Glu) to amino acid number 56 (Asn) of SEQ ID NO:2; (2) amino acid number 97 (Ser) to amino acid number 102 (Gln) of SEQ ID NO:2; (3) amino acid number 272 (Asp) to amino acid number 277 (Asp) of SEQ ID NO:2; (4) amino acid number 339 (Asn) to amino acid number 344 (Glu) of SEQ ID NO:2; and (5) amino acid number 341 (Glu) to amino acid number 346 (Asp) of SEQ ID NO:2. Moreover, zapop2 hydrophilic epitopes as predicted by a Jameson-Wolf plot, e.g., using DNASTAR Protean program (DNASTAR, Inc., Madison, Wis.) can also be determined by one of skill in the art.

Those skilled in the art will recognize that hydrophilicity or hydrophobicity will be taken into account when designing modifications in the amino acid sequence of a zapop2 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp. For example, residues tolerant of substitution could include residues as shown in SEQ ID NO:2. Cysteine residues at positions of SEQ ID NO:2, such as those disclosed herein, are relatively intolerant of substitution.

The identities of essential amino acids can also be inferred from analysis of sequence similarity between RING family proteins with zapop2. Using methods such as “FASTA” analysis described previously, regions of high similarity are identified within a family of proteins and used to analyze amino acid sequence for conserved regions. An alternative approach to identifying a variant zapop2 polynucleotide on the basis of structure is to determine whether a nucleic acid molecule encoding a potential variant zapop2 polynucleotide can hybridize to a nucleic acid molecule having the nucleotide sequence of SEQ ID NO:1, as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699 (1996).

The present invention also includes functional fragments of zapop2 polypeptides and nucleic acid molecules encoding such functional fragments. A “functional” zapop2 or fragment thereof defined herein is characterized by its proliferative or differentiating activity, by its ability to induce or inhibit specialized cell functions, or by its ability to bind specifically to an anti-zapop2 antibody or zapop2 receptor (either soluble or immobilized). Thus, the present invention further provides fusion proteins encompassing: (a) polypeptide molecules comprising one or more of the domains described above; and (b) functional fragments comprising one or more of these domains. The other polypeptide portion of the fusion protein may be contributed by another RING protein or ankyrin repeat containing protein, such as BRCA1, or by a non-native and/or an unrelated secretory signal peptide that facilitates secretion of the fusion protein.

Routine deletion analyses of nucleic acid molecules can be performed to obtain functional fragments of a nucleic acid molecule that encodes a zapop2 polypeptide. As an illustration, DNA molecules having the nucleotide sequence of SEQ ID NO:1 or fragments thereof, can be digested with Bal31 nuclease to obtain a series of nested deletions. These DNA fragments are then inserted into expression vectors in proper reading frame, and the expressed polypeptides are isolated and tested for zapop2 activity, or for the ability to bind anti-zapop2 antibodies or zapop2 receptor. One alternative to exonuclease digestion is to use oligonucleotide-directed mutagenesis to introduce deletions or stop codons to specify production of a desired zapop2 fragment. Alternatively, particular fragments of a zapop2 polynucleotide can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-5A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation 1, Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50:1295 (1995); and Meisel et al., Plant Molec. Biol. 30:1 (1996).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zapop2 DNA and polypeptide sequences can be generated through DNA shuffling as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., that induce proliferation, transformation or apoptosis) can be recovered from the host cells and rapidly sequenced using modem equipment. These methods allow the rapid determination of the importance of individual amino acid residues in a polypeptide of interest, and can be applied to polypeptides of unknown structure.

Using the methods discussed herein, one of ordinary skill in the art can identify and/or prepare a variety of polypeptide fragments or variants of SEQ ID NO:2 or that retain the signal transduction or protein-protein or DNA binding properties of the wild-type zapop2 protein. For example, one can make a zapop2 “protein binding fragment” by preparing a variety of polypeptides that are substantially homologous to the ankyrin repeat region or RING finger domain and retain protein-binding activity of the wild-type zapop2 protein. Such polypeptides may include additional amino acids from, for example, part or all of the N-terminal and C-terminal domains. Such polypeptides may also include additional polypeptide segments as generally disclosed herein such as labels, affinity tags, and the like.

For any zapop2 polypeptide, including variants and fusion proteins, one of ordinary skill in the art can readily generate a fully degenerate polynucleotide sequence encoding that variant using the information set forth in Tables 1 and 2 above.

The zapop2 polypeptides of the present invention, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

In general, a DNA sequence encoding a zapop2 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a zapop2 polypeptide into the secretory pathway of a host cell, a secretory signal sequence (also known as a leader sequence, prepro sequence or pre sequence) is provided in the expression vector. The secretory signal sequence may be that of zapop2, or may be derived from another secreted protein (e.g., t-PA) or synthesized de novo. The secretory signal sequence is operably linked to the zapop2 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

To direct the export of a zapop2 polypeptide from the host cell, the zapop2 DNA is linked to a second DNA segment encoding a secretory peptide, such as a t-PA secretory peptide. To facilitate purification of the secreted receptor polypeptide, a C-terminal extension, such as a poly-histidine tag, substance P, Flag peptide (Hopp et al., Bio/Technology 6:1204-1210, 1988; available from Eastman Kodak Co., New Haven, Conn.) or another polypeptide or protein for which an antibody or other specific binding agent is available, can be fused to the zapop2 polypeptide.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, L. A. and Possee, R. D., The Baculovirus Expression System: A Laboratory Guide, London, Chapman & Hall; O'Reilly, D. R. et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant zapop2 baculovirus utilizes a transposon-based system described by Luckow (Luckow et al., J. Virol. 67:4566-79, 1993). This system, which utilizes transfer vectors, is sold in the Bac-to-Bac™ kit (Life Technologies, Rockville, Md.). This system utilizes a transfer vector, pFastBac1™ (Life Technologies) containing a Tn7 transposon to move the DNA encoding the zapop2 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zapop2. However, pFastBac1™ can be modified to a considerable degree. The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins, M. S. and Possee, R. D., J. Gen. Virol. 71:971-6, 1990; Bonning, B. C. et al., J. Gen. Virol. 75:1551-6, 1994; and, Chazenbalk, G. D., and Rapoport, B., J. Biol. Chem. 270:1543-9, 1995. In such transfer vector constructs, a short or long version of the basic protein promoter can be used. Moreover, transfer vectors can be constructed which replace the native zapop2 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Calif.) can be used in constructs to replace the native zapop2 secretory signal sequence. In addition, transfer vectors can include an in-frame fusion with DNA encoding an epitope tag at the C- or N-terminus of the expressed zapop2 polypeptide, for example, a Glu-Glu epitope tag (Grussenmeyer, T. et al., Proc. Natl. Acad. Sci. 82:7952-4, 1985). Using a technique known in the art, a transfer vector containing zapop2 is transformed into E. coli, and screened for bacmids which contain an interrupted lacZ gene indicative of recombinant baculovirus. The bacmid DNA containing the recombinant baculovirus genome is isolated, using common techniques, and used to transfect Spodoptera frugiperda cells, e.g. Sf9 cells. Recombinant virus that expresses zapop2 is subsequently produced. Recombinant viral stocks are made by methods commonly used the art.

The recombinant virus is used to infect host cells, typically a cell line derived from the fall armyworm, Spodoptera frugiperda. See, in general, Glick and Pasternak, Molecular Biotechnology: Principles and Applications of Recombinant DNA, ASM Press, Washington, D.C., 1994. Another suitable cell line is the High FiveO™ cell line (Invitrogen, San Diego, Calif.) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, Kans.) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King, L. A. and Possee, R. D., ibid.; O'Reilly, D. R. et al., ibid.; Richardson, C. D., ibid.). Subsequent purification of the zapop2 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a zapop2 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zapop2 polypeptides (or chimeric zapop2 polypeptides) can be purified using fractionation and/or conventional purification methods and media. For example, the particular purification methods for TIGR, described in Nguyen, supra., are exemplary, and can be adapted to zapop2 polypeptide by one of ordinary skill in the art using methods described below. An exemplary purification method for protein constructs having an affinity tag such as an N-terminal or C-terminal FLAG tag produced from mammalian cells, such as BHK cells, involves using an antibody to the FLAG tag epitope to purify the zapop2 protein. Conditioned media from BHK cells is sequentially sterile filtered through a 4 inch, 0.2 mM Millipore (Bedford, Mass.) OptiCap capsule filter and a 0.2 mM Gelman (Ann Arbor, Mich.) Supercap 50. The material is then concentrated using an Amicon (Beverly, Mass.) DC 10L concentrator fitted with an A/G Tech (Needham, Mass.) hollow fiber cartridge with a 15 sq. ft. 3000 kDa cutoff membrane. The concentrated material is again sterile-filtered with the Gelman filter as described above. A aliquot of anti-Flag Sepharose (Eastman Kodak, Rochester, N.Y.) is added to the sample for batch adsorption and the mixture is gently agitated on a Wheaton (Millville, N.J.) roller culture apparatus for 18.0 h at 4° C.

The mixture is then poured into a 5.0×20.0 cm Econo-Column (Bio-Rad, Laboratories, Hercules, Calif.) and the gel is washed with 30 column volumes of phosphate buffered saline (PBS). The unretained flow-through fraction is discarded. Once the absorbance of the effluent at 280 nM is less than 0.05, flow through the column is reduced to zero and the anti-Flag Sepharose gel is washed with 2.0 column volumes of PBS containing 0.2 mg/ml of Flag peptide, (SEQ ID NO:4) (Eastman Kodak). After 1.0 hour at 4° C., flow is resumed and the eluted protein is collected. This fraction is referred to as the peptide elution. The anti-Flag Sepharose gel is washed with 2.0 column volumes of 0.1 M glycine, pH 2.5, and the glycine wash is collected separately. The pH of the glycine-eluted fraction is adjusted to 7.0 by the addition of a small volume of 10×PBS and stored at 4° C.

The peptide elution is concentrated to 5.0 ml using a 5,000 molecular weight cutoff membrane concentrator (Millipore, Bedford, Mass.) according to the manufacturer's instructions. The concentrated peptide elution is then separated from free peptide by chromatography on a 1.5×50 cm Sephadex G-50 (Pharmacia, Piscataway, N.J.) column equilibrated in PBS at a flow rate of 1.0 ml/min using a BioCad Sprint HPLC system (PerSeptive BioSystems, Framingham, Mass.). Two-ml fractions are collected and the absorbance at 280 nM is monitored. The first peak of material absorbing at 280 nM and eluting near the void volume of the column is collected.

SDS-PAGE, Western analysis, amino acid analysis and N-terminal sequencing can be done to the purified protein. Protein concentration can be determined by BCA analysis (Pierce, Rockford, Ill.).

Protein purification methods also include, fractionation of samples by ammonium sulfate precipitation and acid or chaotrope extraction. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their biochemical, structural, and biological properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp. 529-39). Within additional embodiments of the invention, a fusion of the polypeptide of interest and an affinity tag (e.g., maltose-binding protein, an immunoglobulin domain) may be constructed to facilitate purification.

Moreover, using methods described in the art, polypeptide fusions, or hybrid zapop2 proteins, are constructed using regions or domains of the inventive zapop2 in combination with those of other RING or ankyrin repeat proteins, or heterologous proteins (Sambrook et al., ibid., Altschul et al., ibid., Picard, Cur. Opin. Biology, 5:511-5, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Fusion proteins can be prepared by methods known to those skilled in the art by preparing each component of the fusion protein and chemically conjugating them. Alternatively, a polynucleotide encoding both components of the fusion protein in the proper reading frame can be generated using known techniques and expressed by the methods described herein. For example, part or all of a domain(s) conferring a biological function may be swapped between zapop2 of the present invention with the functionally equivalent domain(s) from another group member, such as BRCA1. Such domains include, but are not limited to, the N-terminal domain, ankyrin repeat region, individual ankyrin repeats (ankyrin repeat 1-9), central region, C-terminal region, and the RING finger domains described herein. Such fusion proteins would be expected to have a biological functional profile that is the same or similar to polypeptides of the present invention or other known RING protein group members, depending on the fusion constructed. Moreover, such fusion proteins may exhibit other properties as disclosed herein.

Standard molecular biological and cloning techniques can be used to swap the equivalent domains between the zapop2 polypeptide and those polypeptides to which they are fused. Generally, a DNA segment that encodes a domain of interest, e.g., a zapop2 domain described herein, is operably linked in frame to at least one other DNA segment encoding an additional polypeptide (for instance a domain or region from another RING finger protein, such as BRCA1), and inserted into an appropriate expression vector, as described herein. Generally DNA constructs are made such that the several DNA segments that encode the corresponding regions of a polypeptide are operably linked in frame to make a single construct that encodes the entire fusion protein, or a functional portion thereof. For example, a DNA construct would encode from N-terminus to C-terminus a fusion protein comprising an N-terminal region followed by ankyrin repeats, operably connected to a central region, operably connected to a C-terminal region containing at least one RING finger domain; or a C-terminal domain comprising at least one zapop2 RING finger domain fused C-terminally to another protein; or an ankyrin repeat domain fused N-terminally to another protein; and the like. Such fusion proteins can be expressed, isolated, and assayed for activity as described herein. Moreover, such fusion proteins can be used to express and secrete fragments of the zapop2 polypeptide, to be used, for example to inoculate an animal to generate anti-zapop2 antibodies as described herein. For example a secretory signal sequence can be operably linked to an N-terminal region, ankyrin repeat region, central region, C-terminal region containing at least one RING finger domain, or a combination thereof (e.g., operably linked polypeptides comprising the N-terminal region fused to the ankyrin domain, or a central region fused to a C-terminal region containing at least one RING finger domain, and the like, or zapop2 polypeptide fragments described herein), to secrete a fragment of zapop2 polypeptide that can be purified as described herein and serve as an antigen to be inoculated into an animal to produce anti-zapop2 antibodies, as described herein.

Zapop2 polypeptides or fragments thereof may also be prepared through chemical synthesis. zapop2 polypeptides may be monomers or multimers; glycosylated or non-glycosylated; pegylated or non-pegylated; and may or may not include an initial methionine amino acid residue.

Polypeptides of the present invention can also be synthesized by exclusive solid phase synthesis, partial solid phase methods, fragment condensation or classical solution synthesis. Methods for synthesizing polypeptides are well known in the art. See, for example, Merrifield, J. Am. Chem. Soc. 85:2149, 1963; Kaiser et al., Anal. Biochem. 34:595, 1970. After the entire synthesis of the desired peptide on a solid support,the peptide-resin is with a reagent which cleaves the polypeptide from the resin and removes most of the side-chain protecting groups. Such methods are well established in the art.

The activity of molecules of the present invention can be measured using a variety of assays that measure proliferation, morphogensis, apoptosis, or transformation. Such assays are well known in the art.

The polypeptides, nucleic acids and/or antibodies of the present invention can be used in treatment of disorders associated with myocardial infarction, congestive heart failure, hypertrophic cardiomyopathy and dilated cardiomyopathy. Molecules of the present invention may also be useful for limiting infarct size following a heart attack, aiding in recovery after heart transplantation, promoting angiogenesis and wound healing following angioplasty or endarterectomy, to develop coronary collateral circulation, for revascularization in the eye, for complications related to poor circulation such as diabetic foot ulcers, for stroke, following coronary reperfusion using pharmacologic methods, and other indications where angiogenesis is of benefit. Molecules of the present invention may be useful for improving cardiac function, either by inducing cardiac myocyte neogenesis and/or hyperplasia, by inducing coronary collateral development, or by inducing remodeling of necrotic myocardial area. Other therapeutic uses for the present invention include induction of skeletal muscle neogenesis and/or hyperplasia, kidney regeneration and/or for treatment of systemic and pulmonary hypertension.

Zapop2 can be assayed for apoptosis inhibitory activity using the methods of Ambrosini, G. et al., Nature Med. 3:917-921, 1997. Briefly, cDNAs encoding Bcl-2 and zapop2 are cloned into mammalian expression vector pcDNA3 (Invitrogen) and transfected into the IL-3-dependant murine pre-B-cell line, BaF3, using standard molecular biology techniques (Ausubel et al., supra.; Palacios and Steinmetz, Cell 41:727-734, 1985; Mathey-Prevot et al., Mol. Cell. Biol. 6: 4133-4135, 1986; Ascaso, R. et al., Eur. J. Immunol. 24:537-541, 1994). Stable cell lines are selected and cloned by methods disclosed herein, for example by G418 selection. To assess the effect of zapop2 on apoptosis, survival of cells co-expressing Bcl-2 and zapop2 is measured under conditions where apoptosis is normally induced, i.e., when IL-3 is withdrawn from the cell culture medium. Viability can be measured, for, example, by trypan blue staining. Wild-type Baf3 cells, and cells expressing only Bcl-2 are used as positive controls for apoptosis. In the presence of zapop2, inhibition of apoptosis is shown as increased survival of cells expressing zapop2 relative to the control cells.

An in vivo approach for assaying proteins of the present invention involves viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acid (for review, see T. C. Becker et al., Meth. Cell Biol. 43:161-89, 1994; and J. T. Douglas and D. T. Curiel, Science & Medicine 4:44-53, 1997). The adenovirus system offers several advantages: (i) adenovirus can accommodate relatively large DNA inserts; (ii) can be grown to high-titer; (iii) infect a broad range of mammalian cell types; and (iv) can be used with a large number of different promoters including ubiquitous, tissue specific, and regulatable promoters. Also, because adenoviruses are stable in the bloodstream, they can be administered by intravenous injection.

Using adenovirus vectors where portions of the adenovirus genome are deleted, inserts are incorporated into the viral DNA by direct ligation or by homologous recombination with a co-transfected plasmid. In an exemplary system, the essential E1 gene has been deleted from the viral vector, and the virus will not replicate unless the E1 gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, M. et al., J. Virol. 72:2022-2032, 1998; Raper, S. E. et al., Human Gene Therapy 9:671-679, 1998). In addition, deletion of E2b is reported to reduce immune responses (Amalfitano, A. et al., J. Virol. 72:926-933, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh, P. and Perricaudet, M., FASEB J. 11:615-623, 1997.

The adenovirus system can also be used for protein production in vitro. By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (See Garnier et al., Cytotechnol. 15:145-55, 1994). With either protocol, an expressed, secreted heterologous protein can be repeatedly isolated from the cell culture supernatant, lysate, or membrane fractions depending on the disposition of the expressed protein in the cell. Within the infected 293 cell production protocol, non-secreted proteins may also be effectively obtained.

The activation of zapop2 polypeptide can be measured by a silicon-based biosensor microphysiometer which measures the extracellular acidification rate or proton excretion associated with receptor binding and subsequent physiologic cellular responses. An exemplary device is the Cytosensor™ Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell, H. M. et al., Science 257:1906-1912, 1992; Pitchford, S. et al., Meth. Enzymol. 228:84-108, 1997; Arimilli, S. et al., J. Immunol. Meth. 212:49-59, 1998; Van Liefde, I. Et al., Eur. J. Pharmacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including agonists, ligands, or antagonists of the zapop2 polypeptide. Preferably, the microphysiometer is used to measure responses of a zapop2-expressing eukaryotic cell, compared to a control eukaryotic cell that does not express zapop2 polypeptide. Zapop2-expressing eukaryotic cells comprise cells into which zapop2 has been transfected, as described herein, creating a cell that is responsive to zapop2-modulating stimuli; or cells naturally expressing zapop2, such as zapop2-expressing cells derived from spleen, testis, muscle or heart tissue. Differences, measured by a change in extracellular acidification, for example, an increase or diminution in the response of cells expressing zapop2, relative to a control, are a direct measurement of zapop2-modulated cellular responses. Moreover, such zapop2-modulated responses can be assayed under a variety of stimuli. Also, using the microphysiometer, there is provided a method of identifying agonists and antagonists of zapop2 polypeptide, comprising providing cells expressing a zapop2 polypeptide, culturing a first portion of the cells in the absence of a test compound, culturing a second portion of the cells in the presence of a test compound, and detecting a change, for example, an increase or diminution, in a cellular response of the second portion of the cells as compared to the first portion of the cells. The change in cellular response is shown as a measurable change extracellular acidification rate. Antagonists and agonists, including the natural ligand for zapop2 polypeptide, can be rapidly identified using this method.

In view of the tissue distribution observed for zapop2, agonists (including the natural substrate/ cofactor/ etc.) and antagonists have enormous potential in both in vitro and in vivo applications. Compounds identified as zapop2 agonists are useful for stimulating growth of heart, skeletal muscle, immune and hematopoietic cells in vitro and in vivo. For example, zapop2 and agonist compounds are useful as components of defined cell culture media, and may be used alone or in combination with other cytokines and hormones to replace serum that is commonly used in cell culture. Agonists are thus useful in specifically promoting the growth and/or development of cardiac cells, skeletal muscle cells and other cells in culture. Moreover, zapop2 agonist, or antagonist, may be used in vitro in an assay to measure stimulation of colony formation from isolated primary bone marrow cultures. Such assays are well known in the art.

Antagonists are also useful as research reagents for characterizing sites of protein-protein interaction. Inhibitors of zapop2 activity (zapop2 antagonists) include anti-zapop2 antibodies as well as other peptidic and non-peptidic agents (including ribozymes).

Zapop2 can also be used to identify modulators (e.g, agonists or antagonists) of its activity. Test compounds are added to the assays disclosed herein to identify compounds that inhibit or stimulate the activity of zapop2. In addition to those assays disclosed herein, samples can be tested for inhibition/stimulation of zapop2 activity within a variety of assays designed to measure zapop2 binding, dimerization, heterodimerization, DNA binding or the stimulation/inhibition of zapop2-dependent cellular responses. For example, zapop2-expressing cell lines can be transfected with a reporter gene construct that is responsive to a zapop2-stimulated cellular pathway. Reporter gene constructs of this type are known in the art, and will generally comprise a zapop2-DNA response element operably linked to a gene encoding an assay detectable protein, such as luciferase. DNA response elements can include, but are not limited to, cyclic AMP response elements (CRE), hormone response elements (HRE) insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8):1087-94; 1990. Hormone response elements are reviewed in Beato, Cell 56:335-44; 1989. Candidate compounds, solutions, mixtures or extracts or conditioned media from various cell types are tested for the ability to enhance the activity of zapop2 signal transduction as evidenced by a increase in zapop2 stimulation of reporter gene expression. Assays of this type will detect compounds that directly stimulate zapop2 signal transduction activity through binding the upstream receptor or by otherwise stimulating part of the signal cascade in which zapop2 is involved. As such, there is provided a method of identifying agonists of zapop2 polypeptide, comprising providing cells expressing zapop2 responsive to a zapop2 pathway, culturing a first portion of the cells in the absence of a test compound, culturing a second portion of the cells in the presence of a test compound, and detecting a increase in a cellular response of the second portion of the cells as compared to the first portion of the cells. Moreover a third cell, containing the reporter gene construct described above, but not expressing zapop2 polypeptide, can be used as a control cell to assess non-specific, or non-zapop2-mediated, stimulation of the reporter. Agonists are useful to stimulate or increase zapop2 polypeptide function.

Moreover, compounds or other samples can be tested for direct blocking of zapop2 binding to another protein, e.g., a heterodimer described below, using zapop2 tagged with a detectable label (e.g., ¹²⁵I, biotin, horseradish peroxidase, FITC, and the like). Within assays of this type, the ability of a test sample to inhibit the binding of labeled zapop2 to the other protein is indicative of inhibitory activity, which can be confirmed through secondary assays. Proteins used within binding assays may be cellular proteins or isolated, immobilized proteins.

A zapop2 polypeptide can be expressed as a fusion with an immunoglobulin heavy chain constant region, typically an F_{c} fragment, which contains two constant region domains and lacks the variable region. Methods for preparing such fusions are disclosed in U.S. Pat. Nos. 5,155,027 and 5,567,584. Such fusions are typically secreted as multimeric molecules wherein the Fc portions are disulfide bonded to each other and two non-Ig polypeptides are arrayed in closed proximity to each other. Fusions of this type can be used to (any specific uses?, affinity purify ligand, in vitro assay tool, antagonist). For use in assays, the chimeras are bound to a support via the F_{c} region and used in an ELISA format.

A zapop2 ligand-binding polypeptide can also be used for purification of ligand. The polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing ligand are passed through the column one or more times to allow ligand to bind to the receptor polypeptide. The ligand is then eluted using changes in salt concentration, chaotropic agents (guanidine HCl), or pH to disrupt ligand-receptor binding.

An assay system that uses a ligand-binding receptor (or an antibody, one member of a complement/ anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:545-48, 1991; Cunningham et al., Science 245:821-25, 1991).

Zapop2 polypeptides can also be used to prepare antibodies that bind to zapop2 epitopes, peptides or polypeptides. The zapop2 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a zapop2 polypeptide (e.g., SEQ ID NO:2). Polypeptides comprising a larger portion of a zapop2 polypeptide, i.e., from 30 to 10 residues up to the entire length of the amino acid sequence are included. Antigens or immunogenic epitopes can also include attached tags, adjuvants and carriers, as described herein. Suitable antigens include the zapop2 polypeptide encoded by SEQ ID NO:2 from amino acid number 1 (Met) to amino acid number 599 (Val), or a contiguous 9 to 599 amino acid amino acid fragment thereof. Preferred peptides to use as antigens are the N-terminal domain, ankyrin repeat region, central region, C-terminal region and the RING finger domains, disclosed herein, and zapop2 hydrophilic peptides such as those predicted by one of skill in the art from a hydrophobicity plot, determined for example, from a hydrophobicity profile such as that shown in FIG. 1. Zapop2 hydrophilic peptides include peptides comprising amino acid sequences selected from the group consisting of: (1) amino acid number 51 (Glu) to amino acid number 56 (Asn) of SEQ ID NO:2; (2) amino acid number 97 (Ser) to amino acid number 102 (Gln) of SEQ ID NO:2; (3) amino acid number 272 (Asp) to amino acid number 277 (Asp) of SEQ ID NO:2; (4) amino acid number 339 (Asn) to amino acid number 344 (Glu) of SEQ ID NO:2; and (5) amino acid number 341 (Glu) to amino acid number 346 (Asp) of SEQ ID NO:2. Moreover, zapop2 antigenic epitopes as predicted by a Jameson-Wolf plot, e.g., using DNASTAR Protean program (DNASTAR, Inc., Madison, Wis.) serve as preferred antigens including in reference to SEQ ID NO:2: residues 52-58; 68-80; 52-80; 66-80; 97-110; 136-143; 152-157; 136-157; 170-174; 194-200; 205-210; 194-210; 299-313; 365-371; 411-418; 437-444; 448-452; 437-452; 469-473; 515-524; 532-537; 515-537; 590-594. Of particular interest are epitopes within the ankyrin repeat region and the RING finger domain, for example residues 52-80; 136-157; 170-174; 194-200; 299-313; 437-452; 469-473; 515-537 of SEQ ID NO:2. In addition, conserved motifs, and variable regions between conserved motifs of zapop2 are suitable antigens. Antibodies generated from this immune response. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a zapop2 polypeptide or a fragment thereof. The immunogenicity of a zapop2 polypeptide may be increased through the use of an adjuvant, such as alum (aluminum hydroxide) or Freund's complete or incomplete adjuvant. Polypeptides useful for immunization also include fusion polypeptides, such as fusions of zapop2 or a portion thereof with an immunoglobulin polypeptide or with maltose binding protein. The polypeptide μimmunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

Moreover, human antibodies can be produced in transgenic, non-human animals that have been engineered to contain human immunoglobulin genes as disclosed in WIPO Publication WO 98/24893. It is preferred that the endogenous immunoglobulin genes in these animals be inactivated or eliminated, such as by homologous recombination.

Antibodies are considered to be specifically binding if: 1) they exhibit a threshold level of binding activity, and 2) they do not significantly cross-react with related polypeptide molecules. A threshold level of binding is determined if anti-zapop2 antibodies herein specifically bind if they bind to a zapop2 polypeptide, peptide or epitope with an affinity at least 10-fold greater than the binding affinity to control (non-zapop2) polypeptide. It is preferred that the antibodies exhibit a binding affinity (Kₐ) of 10⁶ M^{31 1} or greater, preferably 10⁷ M⁻¹ or greater, more preferably 10⁸ M⁻¹ or greater, and most preferably 10⁹ M⁻¹ or greater. The binding affinity of an antibody can be readily determined by one of ordinary skill in the art, for example, by Scatchard analysis (Scatchard, G., Ann. NY Acad. Sci. 51: 660-672, 1949).

Whether anti-zapop2 antibodies do not significantly cross-react with related polypeptide molecules is shown, for example, by the antibody detecting zapop2 polypeptide but not known related polypeptides using a standard Western blot analysis (Ausubel et al., ibid.). Examples of known related polypeptides are those disclosed in the prior art, such as known orthologs, and paralogs, and similar known members of a protein family or group, Screening can also be done using non-human zapop2, and zapop2 mutant polypeptides. Moreover, antibodies can be “screened against” known related polypeptides, to isolate a population that specifically binds to the inventive zapop2 polypeptides. For example, antibodies raised to zapop2 are adsorbed to related polypeptides adhered to insoluble matrix; antibodies specific to zapop2 will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental Immunology, Paul (eds.), Raven Press, 1993; Getzoff et al., Adv. in Immunol. 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984. Specifically binding anti-zapop2 antibodies can be detected by a number of methods in the art, and disclosed below.

A variety of assays known to those skilled in the art can be utilized to detect antibodies which specifically bind to zapop2 proteins or peptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmunoprecipitation, enzyme-linked immunosorbent assay (ELISA), dot blot or Western blot assay, inhibition or competition assay, and sandwich assay. In addition, antibodies can be screened for binding to wild-type versus mutant zapop2 protein or polypeptide.

Alternative techniques for generating or selecting antibodies useful herein include in vitro exposure of lymphocytes to zapop2 protein or peptide, and selection of antibody display libraries in phage or similar vectors (for instance, through use of immobilized or labeled zapop2 protein or peptide). Genes encoding polypeptides having potential zapop2 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the zapop2 sequences disclosed herein to identify proteins which bind to zapop2. These “binding polypeptides” which interact with zapop2 polypeptides can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for blocking interaction between ligand and receptor, or viral binding to a receptor. The binding polypeptides can also be used for diagnostic assays for determining circulating levels of zapop2 polypeptides; for detecting or quantitating soluble zapop2 polypeptides as marker of underlying pathology or disease. These binding polypeptides can also act as zapop2 “antagonists” to block zapop2 binding and signal transduction in vitro and in vivo. These anti-zapop2 binding polypeptides would be useful for inhibiting zapop2 activity or protein-binding.

Antibodies to zapop2 may be used for tagging cells that express zapop2; for isolating zapop2 by affinity purification; for diagnostic assays for determining circulating levels of zapop2 polypeptides; for detecting or quantitating soluble zapop2 as marker of underlying pathology or disease; in analytical methods employing FACS; for screening expression libraries; for generating anti-idiotypic antibodies; and as neutralizing antibodies or as antagonists to block zapop2 activity in vitro and in vivo. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to zapop2 or fragments thereof may be used in vitro to detect denatured zapop2 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zapop2 polypeptides or anti-zapop2 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/ anticomplementary pair, where the other member is bound to the polypeptide or antibody portion. For these purposes, biotin/streptavidin is an exemplary complementary/ anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, if the polypeptide has multiple functional domains (i.e., an activation domain or a ligand binding domain, plus a targeting domain), a fusion protein including only the targeting domain may be suitable for directing a detectable molecule, a cytotoxic molecule or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/ cytotoxic molecule conjugates.

In another embodiment, zapop2-cytokine fusion proteins or antibody-cytokine fusion proteins can be used for enhancing in vivo killing of target tissues (for example, blood and bone marrow cancers), if the zapop2 polypeptide or anti-zapop2 antibody targets the hyperproliferative blood or bone marrow cell (See, generally, Homick et al., Blood 89:4437-47, 1997). They described fusion proteins enable targeting of a cytokine to a desired site of action, thereby providing an elevated local concentration of cytokine. Suitable zapop2 polypeptides or anti-zapop2 antibodies target an undesirable cell or tissue (i.e., a tumor or a leukemia), and the fused cytokine mediated improved target cell lysis by effector cells. Suitable cytokines for this purpose include interleukin 2 and granulocyte-macrophage colony-stimulating factor (GM-CSF), for instance.

Moreover, such conjugates can be used as diagnostics for human disease, or for detection of specific tissues in which zapop2 is expressed. For example, labeled conjugates and anti-zapop2 antibodies can be used to identify diseased tissues, cells, cancers, necrosis, and the like, that over-express or under-express zapop2 relative to a normal non-diseased control. For example, labeled conjugates and anti-zapop2 antibodies can be used to identify tissues wherein zapop2 is specifically expressed, such as heart and skeletal muscle. Histological methods known in the art, and other assays described herein can be used with these conjugates to identify diseased tissues.

In yet another embodiment, if the zapop2 polypeptide or anti- zapop2 antibody targets vascular cells or tissues, such polypeptide or antibody may be conjugated with a radionuclide, and particularly with a beta-emitting radionuclide, to reduce restenosis. Such therapeutic approach poses less danger to clinicians who administer the radioactive therapy. For instance, iridium-192 impregnated ribbons placed into stented vessels of patients until the required radiation dose was delivered showed decreased tissue growth in the vessel and greater luminal diameter than the control group, which received placebo ribbons. Further, revascularisation and stent thrombosis were significantly lower in the treatment group. Similar results are predicted with targeting of a bioactive conjugate containing a radionuclide, as described herein.

The bioactive polypeptide or antibody conjugates described herein can be delivered intravenously, intraarterially or intraductally, or may be introduced locally at the intended site of action.

As a polypeptide containing several ankyrin repeats, zapop2 can be involved with interactions in the cytoskeleton, focal adhesions, spectrin binding, and the like. Proteins that affect the cytoskeleton are important in regulating or modulating cytoskeletal reorganization, gene transcription in a cell, cytoskeletal organization and cell division, cell motility, transformation, or invasiveness. As such, proteins affecting the cytoskeletal arrangement can be implicated in a variety of cancers where increased cell motility, transformation, or invasiveness are key steps in tumor formation. Thus zapop2, its agonists and antagonists can be important in diagnostic and therapeutic applications for cancer.

The activity of molecules of the present invention can also be measured using a variety of assays that measure, for example, cell motility, adhesion and invasion in vitro and metastasis in vivo. Such assays are known in the art. For example, motility assays in NIH 3T3 cells, mouse keratinocytes, and epithelial cells are described in Takiashi, K. et al., Mol. Cell Biol. 13:72-79, 1993; Takiashi, K. et al., Oncogene 5:273-278, 1994; Ridley, A. J. et al., Mol. Cell Biol. 15:1110-1122, 1995; and Keely, P. J. et al., Nature 360:632-636, 1997. For review and application of in vitro invasion assays; for example, using hepatoma or lymphoma cells invasion through mesothelial or fibroblast cell monolayers, phagokenesis and wound healing assays. For example, see Yoshioka, K. et al., FEBS Lett. 372:25-28, 1995; Wang, W. Z., and Ron D. Science 272:1347-1349, 1996; Habets, G. Cell 77:537-549, 1994; and Michiels, F. et al., Nature 375:338-340, 1995; Michiels, F. and Collard, J. G., Biochem. Soc. Symp. 65:215-146, 1999; and Keely, P. J. supra. Moreover, in vivo metastasis assays can be used to assess zapop2 polypeptide, expression, agonist or antagonist activity in vivo in mice (Verschueren, H. Eur. J. Cell Biol. 73:182-187, 1997). Cell adhesion can be assessed by the adherence or non-adherence of normally adherent cell lines to cell culture dishes, amongst other assays known in the art.

Moreover, the activity of molecules of the present invention can also be measured using a variety of assays that measure cytoskeletal reorganization. Such assays are well known in the art. For example, effects of zapop2 on membrane ruffling can be assessed in Swiss 3T3 cells (Ridley, A. J. Cell 70:401-410, 1992). Actin polymerization and cytoskeletal rearrangement including assessment of actin stress fibers, focal complexes, lamellipodia and filopodia, can be assessed by various means including immunofluorescence, and time-lapse imaging amongst other known methods (Symons, M. et al., Cell 84:723-734, 1996; Nobes, C. D., and Hall, A., Cell 81:53-62, 1995; Burbelo, P. D. et al., Proc. Natl. Acad. Sci. U.S.A 96:9083-9088, 1999; Aspenstrom, P. Exper. Cell. Res. 246:20-25, 1999; Gallo, G., and Letourneau, P. C., Current Biol. 8:R80-R82, 1998; and Miki, H. et al., Nature 391:93-96, 1998). Moreover, the activity and effect of zapop2 on tumor progression and metastasis can be measured in vivo. Several syngeneic mouse models have been developed to study the influence of polypeptides, compounds or other treatments on tumor progression. In these models, tumor cells passaged in culture are implanted into mice of the same strain as the tumor donor. The cells will develop into tumors having similar characteristics in the recipient mice, and metastasis will also occur in some of the models. Appropriate tumor models for our studies include the Lewis lung carcinoma (ATCC No. CRL-1642) and B16 melanoma (ATCC No. CRL-6323), amongst others. These are both commonly used tumor lines, syngeneic to the C57BL6 mouse, that are readily cultured and manipulated in vitro. Tumors resulting from implantation of either of these cell lines are capable of metastasis to the lung in C57BL6 mice. The Lewis lung carcinoma model has recently been used in mice to identify an inhibitor of angiogenesis (O'Reilly MS, et al. Cell 79: 315-328,1994). C57BL6/J mice are treated with an experimental agent either through daily injection of recombinant protein, agonist or antagonist or a one time injection of recombinant adenovirus. Three days following this treatment, 10⁵ to 10⁶ cells are implanted under the dorsal skin. Alternatively, the cells themselves may be infected with recombinant adenovirus, such as one expressing zapop2, before implantation so that the protein is synthesized at the tumor site or intracellularly, rather than systemically. The mice normally develop visible tumors within 5 days. The tumors are allowed to grow for a period of up to 3 weeks, during which time they may reach a size of 1500-1800 mm³ in the control treated group. Tumor size and body weight are carefully monitored throughout the experiment. At the time of sacrifice, the tumor is removed and weighed along with the lungs and the liver. The lung weight has been shown to correlate well with metastatic tumor burden. As an additional measure, lung surface metastases are counted. The resected tumor, lungs and liver are prepared for histopathological examination, immunohistochemistry, and in situ hybridization, using methods known in the art and described herein. The influence of the expressed polypeptide in question, e.g., zapop2, on the ability of the tumor to recruit vasculature and undergo metastasis can thus be assessed. In addition, aside from using adenovirus, the implanted cells can be transiently transfected with zapop2. Use of stable zapop2 transfectants as well as use of induceable promoters to activate zapop2 expression in vivo are known in the art and can be used in this system to assess zapop2 induction of metastasis. For general reference see, O'Reilly MS, et al. Cell 79:315-328, 1994; and Rusciano D, et al. Murine Models of Liver Metastasis. Invasion Metastasis 14:349-361, 1995.

Differentiation is a progressive and dynamic process, beginning with pluripotent stem cells and ending with terminally differentiated cells. Pluripotent stem cells that can regenerate without commitment to a lineage express a set of differentiation markers that are lost when commitment to a cell lineage is made. Progenitor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products, and receptors. The stage of a cell population's differentiation is monitored by identification of markers present in the cell population. Myocytes, osteoblasts, adipocytes, chrondrocytes, fibroblasts and reticular cells are believed to originate from a common mesenchymal stem cell (Owen et al., Ciba Fdn. Symp. 136:42-46, 1988). Markers for mesenchymal stem cells have not been well identified (Owen et al., J. of Cell Sci. 87:731-738, 1987), so identification is usually made at the progenitor and mature cell stages. The novel polypeptides of the present invention may be useful for studies to isolate mesenchymal stem cells and myocyte or other progenitor cells, both in vivo and ex vivo.

There is evidence to suggest that factors that stimulate specific cell types down a pathway towards terminal differentiation or dedifferentiation affect the entire cell population originating from a common precursor or stem cell. Thus, the present invention includes stimulating or inhibiting the proliferation of myocytes, smooth muscle cells, osteoblasts, adipocytes, chrondrocytes and endothelial cells. Molecules of the present invention for example, may while stimulating proliferation or differentiation of cardiac myocytes, inhibit proliferation or differentiation of adipocytes, by virtue of the affect on their common precursor/stem cells. Thus molecules of the present invention may have use in inhibiting chondrosarcomas, atherosclerosis, restenosis and obesity.

Assays measuring differentiation include, for example, measuring cell markers associated with stage-specific expression of a tissue, enzymatic activity, functional activity or morphological changes (Watt, FASEB, 5:281-284, 1991; Francis, Differentiation 57:63-75, 1994; Raes, Adv. Anim. Cell Biol. Technol. Bioprocesses, 161-171, 1989; all incorporated herein by reference). Alternatively, zapop2 polypeptide itself can serve as an additional cell-surface marker associated with stage-specific expression of a tissue. As such, direct measurement of zapop2 polypeptide, or its loss of expression in a tissue as it differentiates, can serve as a marker for differentiation of tissues.

Similarly, direct measurement of zapop2 polypeptide, or its loss of expression in a tissue can be determined in a tissue or cells as they undergo tumor progression. The gain or loss of expression of zapop2 in a pre-cancerous or cancerous condition, in comparison to normal tissue, can serve as a diagnostic for transformation, invasion and metastasis in tumor progression. As such, knowledge of a tumor's stage of progression or metastasis will aid the physician in choosing the most proper therapy, or aggressiveness of treatment, for a given individual cancer patient. Methods of measuring gain and loss of expression (of either mRNA or protein) are well known in the art and described herein and can be applied to zapop2 expression. For example, appearance or disappearance of polypeptides that regulate cell motility can be used to aid diagnosis and prognosis of prostate cancer (Banyard, J. and Zetter, B. R., Cancer and Metast. Rev. 17:449-458, 1999). As a possible effector of cell motility or not, zapop2 gain or loss of expression may also serve as a diagnostic for cancers. Moreover, analogous to the prostate specific antigen (PSA), as a naturally-expressed testicular marker, increased levels of zapop2 polypeptides, or anti-zapop2 antibodies in a patient, relative to a normal control can be indicative of skeletal muscle, or heart tissue cancers (See, e.g., Mulders, TMT, et al., Eur. J. Surgical Oncol. 16:37-41, 1990). Moreover, as zapop2 expression appears to be restricted to specific human tissues, lack of zapop2 expression in those tissues or strong zapop2 expression in tissues where zapop2 is not normally expressed, would serve as a diagnostic of an abnormality in the cell or tissue type, of invasion or metastasis of cancerous skeletal muscle or heart tissues into non-muscle or heart tissue, and could aid a physician in directing further testing or investigation, or aid in directing therapy.

In addition, as zapop2 is skeletal muscle and heart-specific, polynucleotide probes, anti-zapop2 antibodies, and detection the presence of zapop2 polypeptides in tissue can be used to assess whether these tissues are present, for example, after surgery involving the excision of a diseased or cancerous skeletal muscle or heart tissue. As such, the polynucleotides, polypeptides, and antibodies of the present invention can be used as an aid to determine whether all such tissue is excised after surgery, for example, after surgery for cancer. In such instances, it is especially important to remove all potentially diseased tissue to maximize recovery from the cancer, and to minimize recurrence. Reciprocally, assessing the presence of non-diseased heart or skeletal muscle tissue wherein a tumor from another tissues source has invaded, can aid a surgeon in determining whether a cancer has invaded that tissue. Preferred embodiments include fluorescent, radiolabeled, or calorimetrically labeled anti-zapop2 antibodies and zapop2 polypeptide binding partners, that can be used histologically or in situ.

Molecules of the present invention can be used to identify and isolate proteins that bind or heterodimerize with zapop2. For example, proteins and polypeptides of the present invention can be immobilized on a column and cell lysate preparations run over the column (Immobilized Affinity Ligand Techniques, Hermanson et al., eds., Academic Press, San Diego, Calif., 1992, pp. 195-202). Proteins and polypeptides can also be radiolabeled (Methods in Enzymol., vol. 182, “Guide to Protein Purification”, M. Deutscher, ed., Acad. Press, San Diego, 1990, 721-37) or photoaffinity labeled (Brunner et al., Ann. Rev. Biochem. 62:483-514, 1993 and Fedan et al., Biochem. Pharmacol. 33:1167-80, 1984) and specific cellular proteins can be identified. For example, a zapop2 protein-binding polypeptide, such as the ankyrin repeat region or RING finger domain disclosed herein, can also be used for purification of a heterodimeric protein to which zapop2 binds. The zapop2 protein-binding polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids or cell lysates containing the heterodimeric protein are passed through the column one or more times to allow the heterodimeric protein to bind to the zapop2 protein-binding polypeptide. The heterodimeric protein is then eluted using changes in salt concentration, chaotropic agents (guanidine HCl ), or pH to disrupt protein-protein binding.

The molecules of the present invention will be useful to identify cancers that over-express zapop2 or express mutant forms of the polypeptide. The polypeptides, nucleic acid and/or antibodies of the present invention can be used in treatment of disorders associated with cancer. The molecules of the present invention can be used to identify, modulate, treat and/or prevent development of pathological conditions in such diverse tissue as heart and skeletal muscle. In particular, certain cancers may be amenable to such diagnosis, treatment or prevention.

For example, mutations in the RING finger domain in the breast cancer susceptibility gene, BRCA1, correspond with inability of the protein to dimerize, and are linked to certain breast cancers (Brzovic, P. S. et al., supra. 273). Similarly, as another member of the RING finger group, zapop2 mutations or elevated expression may be associated with specific cancers. Thus, zapop2 polynucleotides and antibodies, described herein, can be used to identify such cancers, serving as a diagnostic for cancer susceptibility, as well as treat through gene therapy.

Using methods known in the art, antibodies to zapop2 and zapop2 polynucleotides can be radiolabeled, fluorescent or chemically labeled and used in histological assays to detect elevated zapop2 present in biopsies. Zapop2 antibodies and zapop2 polynucleotides of the present invention are useful for measuring changes in levels of expression of zapop2 polypeptides. Because zapop2 expression is restricted to specific tissues (i.e., heart and skeletal muscle, with low expression in other tissues), changes in expression levels could be used to monitor metabolism within these tissues. For example, increases in expression and/or transcription of zapop2 polypeptides and polynucleotides, may be predictive for increased cell proliferation of tumor cells. Furthermore, expression of zapop2 in tissue not normally expressing zapop2, for example, ovary and lung, may be indicative of metastasis of tumor cells.

Zapop2 may be demonstrated to be expressed differentially in certain epithelial tissues and carcinomas, particularly in lung, stomach, colon, esophagus, or intestine. Differential expression is the transient expression, or lack thereof, of specific genes, proteins or other phenotypic properties (known as differentiation markers) that occur during the progress of maturation in a cell or tissue. A set of differentiation markers is defined as one or more phenotypic properties that can be identified and are specific to a particular cell type. Thus, pluripotent stem cells that can regenerate without commitment to a lineage express a set of differentiation markers that are lost when commitment to a cell lineage is made. Precursor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products and receptors. Thus, Zapop2 expression can be used as a differentiation marker in normal and tumor tissues to determine the stage of the tumor or maturity of a cell.

A set of differentiation markers is defined as one or more phenotypic properties that can be identified and are specific to a particular cell type. Differentiation markers are transiently exhibited at various stages of cell lineage. Pluripotent stem cells that can regenerate without commitment to a lineage express a set of differentiation markers that are lost when commitment to a cell lineage is made. Precursor cells express a set of differentiation markers that may or may not continue to be expressed as the cells progress down the cell lineage pathway toward maturation. Differentiation markers that are expressed exclusively by mature cells are usually functional properties such as cell products, enzymes to produce cell products and receptors. The activity of molecules of the present invention can be measured using a variety of assays that measure proliferation and/or differentiation of specific cell types, regulation of second messenger levels and chemokine and neurotransmitter release. Such assays are well known in the art and described herein.

Additional methods using probes or primers derived, for example, from the nucleotide sequences disclosed herein can also be used to detect zapop2 expression in a patient sample, such as a tumor biopsy, stomach, lung, blood, saliva, tissue sample, or the like. For example, probes can be hybridized to tumor tissues and the hybridized complex detected by in situ hybridization. Zapop2 sequences can also be detected by PCR amplification using cDNA generated by reverse translation of sample mRNA as a template (PCR Primer A Laboratory Manual, Dieffenbach and Dveksler, eds., Cold Spring Harbor Press, 1995). When compared with a normal control, both increases or decreases of zapop2 expression in a patient sample, relative to that of a control, can be monitored and used as an indicator or diagnostic for disease.

Polynucleotides encoding zapop2 polypeptides are useful within gene therapy applications where it is desired to increase or inhibit zapop2 activity. If a mammal has a mutated or absent zapop2 gene, the zapop2 gene can be introduced into the cells of the mammal. In one embodiment, a gene encoding a zapop2 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV 1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zapop2 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Pat. Publication No. WO 95/07358, published Mar. 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zapop2 gene transcription, such as to inhibit cell proliferation in vivo. Polynucleotides that are complementary to a segment of a zapop2-encoding polynucleotide (e.g., a polynucleotide as set forth in SEQ ID NO:1) are designed to bind to zapop2-encoding mRNA and to inhibit translation of such mRNA. Such antisense polynucleotides are used to inhibit expression of zapop2 polypeptide-encoding genes in cell culture or in a subject.

The present invention also provides reagents which will find use in diagnostic applications. For example, the zapop2 gene, a probe comprising zapop2 DNA or RNA or a subsequence thereof can be used to determine if the zapop2 gene is present on chromosome 1 or if a mutation has occurred. Zapop2 is located at the 1p36 region of chromosome 1 (See, Example 3). Detectable chromosomal aberrations at the zapop2 gene locus include, but are not limited to, aneuploidy, gene copy number changes, translocation, loss of heterozygosity (LOH), insertions, deletions, restriction site changes and rearrangements. Such aberrations can be detected using polynucleotides of the present invention by employing molecular genetic techniques, such as restriction fragment length polymorphism (RFLP) analysis, fluorescence in situ hybridization methods, short tandem repeat (STR) analysis employing PCR techniques, and other genetic linkage analysis techniques known in the art (Sambrook et al., ibid.; Ausubel et. al., ibid.; Marian, Chest 108:255-65, 1995).

The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have.

The zapop2 gene is located at the 1p36 region of chromosome 1. One of skill in the art would recognize that chromosomal abnormalities such as loss, translocation, and rearrangement in 1p36 are associated with human diseases, such as cancer. As such, zapop2 polynucleotide probes within 1p36 can be useful in detecting chromosomal abnormalities associated with human disease. For example, several cancer susceptibility markers map to defects in 1p36, as there appears to be tumor suppressors located there: Breast cancer ductal 2 and 1 (deletions, duplications, structural rearrangements in chromosome 1, and potential link with rh locus) (e.g., see, Genuardi, M. et al., Am. J. Hum. Genet. 45:73-82, 1989; Kovacs, G. Int. J. Cancer 12:688-694, 1978; and Rodgers, C. S. et al., Cancer Genet. Cytogenet. 35:95-119, 1984); prostate and brain cancer (LOH); neuroblastoma and rhabdomyosarcoma (see, below); melanoma (see below) and more. For example, loss of heterozygosity between 1p36.1 and 1p36.3 and other rearrangements in this region are linked to familial neuroblastoma (for example, see Fong, C. -T. et al., Proc. Nat. Acad. Sci. 86:3753-3757, 1989). In addition translocations at the PAX7 locus at 1p36.2-p36.12 and chromosome 13 are associated with rhabdomyosarcoma type 2. The zapop2 polynucleotide probes of the present invention can be used to detect abnormalities or genotypes associated with chromosomal translocations and loss, such as those that are implicated in neuroblastoma, rhabdomyosarcoma, or other cancers to identify heterozygous carriers of a defective 1p36 locus for genetic testing. In addition, zapop2 polynucleotide probes can be used to detect abnormalities or genotypes associated with cutaneous malignant melanoma 1 (1p36), wherein loss of heterozygosity is prevalent in the familial disease (e.g., see Bale, S. J. et al., Cytogenet. Cell Genet. 51:956, 1989; and Dracopoli, N. C. et al., Am. J. Hum. Genet. 45(sup.):A19, 1989). Moreover, the rhesus blood group maps to 1p36.2-p34. The zapop2 polynucleotide probes of the present invention can be used to detect rh polymorphisms in blood and other biological samples for use in transplantation typing, forensics, and the like, or to detect Rh-null disease. In addition, zapop2 polynucleotide probes can be used to detect abnormalities or genotypes associated with systemous lupus erythmatosus (SLE) and glomerulonephritis resulting from complement component C1q (1p36.3-p34.1) deficiency. Further, zapop2 polynucleotide probes can be used to detect abnormalities or genotypes associated with 3-hydroxy-3methylglutaryl-coenzyme A lyase (HMG-CoA lyase) deficiency which maps 1pter-p33, and is associated with Reye-like syndrome, and metabolic acidosis, hypoglycemia, and early death. Further, zapop2 polynucleotide probes can be used to detect abnormalities or genotypes associated with a congenital muscular dystrophy called rigid spine muscular dystrophy (RSMD) which maps 1p36-p35, however there is no associated gene. As zapop2 is highly expressed in skeletal muscle, and is localized to 1p36, defects therein may be associated with RSMD. Moreover, amongst other genetic loci, those for salivary carbonic anhydrase (1p36.33-p36.22), hypophosphatasia (1p36.1-p34), Bartter's Syndrome type III (kidney chloride channels A and B) (1p36), Charcot-Marie-Tooth disease (1p36-p35) all manifest or potentially manifest themselves in human disease states as well as map to this region of the human genome. See the Online Mendellian Inheritance of Man (OMIM) gene map, and references therein, for this region of chromosome 1 on a publicly available WWW server (http://www3.ncbi.nlm.nih.gov/htbin-post/Omim/getmap?chromosome=1p36). All of these serve as possible candidate genes for an inheritable disease which show linkage to the same chromosomal region as the zapop2 gene.

Similarly, defects in the zapop2 locus itself may result in a heritable human disease state. For example, in rigid spine muscular dystrophy (RSMD), described above, patients experience muscle atrophy, changes in muscle biopsy, rigidity of the spine. As zapop2 is highly expressed in both heart and skeletal muscle, defects in zapop2 polypeptide may directly or indirectly cause symptoms of this disease, for example, by improperly binding with a heterodimeric protein important for normal cell function. The zapop2 gene (1p36) is located in a chromosomal region involved in human disease, as discussed above. Moreover, one of skill in the art would appreciate that defects in RING finger proteins (e.g., BRCA1) are known to cause disease states or increase susceptibility to disease in humans. Thus, similarly, defects in zapop2 can cause a disease state or susceptibility to disease. As, zapop2 is a RING protein in a chromosomal hot spot for aberrations involved in numerous cancers, the molecules of the present invention could also be directly involved in cancer formation or metastasis. Molecules of the present invention, such as the polypeptides, antagonists, agonists, polynucleotides and antibodies of the present invention would aid in the detection, diagnosis prevention, and treatment associated with a zapop2 genetic defect.

A diagnostic could assist physicians in determining the type of disease and appropriate associated therapy, or assistance in genetic counseling. As such, the inventive anti-zapop2 antibodies, polynucleotides, and polypeptides can be used for the detection of zapop2 polypeptide, mRNA or anti-zapop2 antibodies, thus serving as markers and be directly used for detecting or genetic diseases or cancers, as described herein, using methods known in the art and described herein. Further, zapop2 polynucleotide probes can be used to detect abnormalities or genotypes associated with chromosome 1p36 deletions and translocations associated with human diseases, other translocations involved with malignant progression of tumors or other 1p36 mutations, which are expected to be involved in chromosome rearrangements in malignancy; or in other cancers, or in spontaneous abortion. Similarly, zapop2 polynucleotide probes can be used to detect abnormalities or genotypes associated with chromosome 1p36 trisomy and chromosome loss associated with human diseases. Thus, zapop2 polynucleotide probes can be used to detect abnormalities or genotypes associated with these defects.

As discussed above, defects in the zapop2 gene itself may result in a heritable human disease state. Molecules of the present invention, such as the polypeptides, antagonists, agonists, polynucleotides and antibodies of the present invention would aid in the detection, diagnosis prevention, and treatment associated with a zapop2 genetic defect. In addition, zapop2 polynucleotide probes can be used to detect allelic differences between diseased or non-diseased individuals at the zapop2 chromosomal locus. As such, the zapop2 sequences can be used as diagnostics in forensic DNA profiling.

In general, the diagnostic methods used in genetic linkage analysis, to detect a genetic abnormality or aberration in a patient, are known in the art. Analytical probes will be generally at least 20 nt in length, although somewhat shorter probes can be used (e.g., 14-17 nt). PCR primers are at least 5 nt in length, preferably 15 or more, more preferably 20-30 nt. For gross analysis of genes, or chromosomal DNA, a zapop2 polynucleotide probe may comprise an entire exon or more. Exons are readily determined by one of skill in the art by comparing zapop2 sequences (SEQ ID NO:1) with the human genomic DNA for zapop2. In general, the diagnostic methods used in genetic linkage analysis, to detect a genetic abnormality or aberration in a patient, are known in the art. Most diagnostic methods comprise the steps of (a) obtaining a genetic sample from a potentially diseased patient, diseased patient or potential non-diseased carrier of a recessive disease allele; (b) producing a first reaction product by incubating the genetic sample with a zapop2 polynucleotide probe wherein the polynucleotide will hybridize to complementary polynucleotide sequence, such as in RFLP analysis or by incubating the genetic sample with sense and antisense primers in a PCR reaction under appropriate PCR reaction conditions; (iii) Visualizing the first reaction product by gel electrophoresis and/or other known method such as visualizing the first reaction product with a zapop2 polynucleotide probe wherein the polynucleotide will hybridize to the complementary polynucleotide sequence of the first reaction; and (iv) comparing the visualized first reaction product to a second control reaction product of a genetic sample from wild type patient. A difference between the first reaction product and the control reaction product is indicative of a genetic abnormality in the diseased or potentially diseased patient, or the presence of a heterozygous recessive carrier phenotype for a non-diseased patient, or the presence of a genetic defect in a tumor from a diseased patient, or the presence of a genetic abnormality in a fetus or pre-implantation embryo. For example, a difference in restriction fragment pattern, length of PCR products, length of repetitive sequences at the zapop2 genetic locus, and the like, are indicative of a genetic abnormality, genetic aberration, or allelic difference in comparison to the normal wild type control. Controls can be from unaffected family members, or unrelated individuals, depending on the test and availability of samples. Genetic samples for use within the present invention include genomic DNA, mRNA, and cDNA isolated form any tissue or other biological sample from a patient, such as but not limited to, blood, saliva, semen, embryonic cells, amniotic fluid, and the like. The polynucleotide probe or primer can be RNA or DNA, and will comprise a portion of SEQ ID NO:1, the complement of SEQ ID NO:1, or an RNA equivalent thereof. Such methods of showing genetic linkage analysis to human disease phenotypes are well known in the art. For reference to PCR based methods in diagnostics see see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

Aberrations associated with the zapop2 locus can be detected using nucleic acid molecules of the present invention by employing standard methods for direct mutation analysis, such as restriction fragment length polymorphism analysis, short tandem repeat analysis employing PCR techniques, amplification-refractory mutation system analysis, single-strand conformation polymorphism detection, RNase cleavage methods, denaturing gradient gel electrophoresis, fluorescence-assisted mismatch analysis, and other genetic analysis techniques known in the art (see, for example, Mathew (ed.), Protocols in Human Molecular Genetics (Humana Press, Inc. 1991), Marian, Chest 108:255 (1995), Coleman and Tsongalis, Molecular Diagnostics (Human Press, Inc. 1996), Elles (ed.) Molecular Diagnosis of Genetic Diseases (Humana Press, Inc. 1996), Landegren (ed.), Laboratory Protocols for Mutation Detection (Oxford University Press 1996), Birren et al. (eds.), Genome Analysis, Vol. 2: Detecting Genes (Cold Spring Harbor Laboratory Press 1998), Dracopoli et al. (eds.), Current Protocols in Human Genetics (John Wiley & Sons 1998), and Richards and Ward, “Molecular Diagnostic Testing,” in Principles of Molecular Medicine, pages 83-88 (Humana Press, Inc. 1998)). Direct analysis of an zapop2 gene for a mutation can be performed using a subject's genomic DNA. Methods for amplifying genomic DNA, obtained for example from peripheral blood lymphocytes, are well-known to those of skill in the art (see, for example, Dracopoli et al. (eds.), Current Protocols in Human Genetics, at pages 7.1.6 to 7.1.7 (John Wiley & Sons 1998)).

Mice engineered to express the zapop2 gene, referred to as “transgenic mice,” and mice that exhibit a complete absence of zapop2 gene function, referred to as “knockout mice,” may also be generated (Snouwaert et al., Science 257:1083, 1992; Lowell et al., Nature 366:740-42, 1993; Capecchi, M. R., Science 244: 1288-1292, 1989; Palmiter, R. D. et al. Annu Rev Genet. 20: 465-499, 1986). For example, transgenic mice that over-express zapop2, either ubiquitously or under a tissue-specific or tissue-restricted promoter can be used to ask whether over-expression causes a phenotype. For example, over-expression of a wild-type zapop2 polypeptide, polypeptide fragment or a mutant thereof may alter normal cellular processes, resulting in a phenotype that identifies a tissue in which zapop2 expression is functionally relevant and may indicate a therapeutic target for the zapop2, its agonists or antagonists. For example, preferred transgenic mice to engineer are ones that over-expresses the entire zapop2 polypeptide, or polypeptides comprising the N-terminal region containing the ankyrin repeats, or the RING-finger domain, as described herein. Moreover, such over-expression may result in a phenotype that shows similarity with human diseases. Similarly, knockout zapop2 mice can be used to determine where zapop2 is absolutely required in vivo. The phenotype of knockout mice is predictive of the in vivo effects of that a zapop2 antagonist, such as those described herein, may have. The human zapop2 cDNA can be used to isolate murine zapop2 mRNA, cDNA and genomic DNA, which are subsequently used to generate knockout mice. These transgenic and knockout mice may be employed to study the zapop2 gene and the protein encoded thereby in an in vivo system, and can be used as in vivo models for corresponding human or animal diseases (such as those in commercially viable animal populations). The mouse models of the present invention are particularly relevant as tumor models for the study of cancer biology and progression. Such models are useful in the development and efficacy of therapeutic molecules used in human cancers. Because increases in zapop2 expression, as well as decreases in zapop2 expression are associated with specific human cancers, both transgenic mice and knockout mice would serve as useful animal models for cancer. Moreover, in a preferred embodiment, zapop2 transgenic mouse can serve as an animal model for specific tumors, particularly skeletal muscle cancer. Moreover, transgenic mice expression of zapop2 antisense polynucleotides or ribozymes directed against zapop2, described herein, can be used analogously to transgenic mice described above.

The invention is further illustrated by the following non-limiting examples.

### EXAMPLES

### Example 1

### Identification of zapop2

### Using an EST Sequence to Obtain Full-length zapop2

Scanning of translated DNA databases using RING finger domain as a query resulted in identification of an expressed sequence tag (EST) sequence. The initial EST sequence was contained in a plasmid, and contained a partial sequence.

An arrayed human salivary gland cDNA/plasmid library was screened by PCR for zapop2 clones using primers ZC15,399 (SEQ ID NO:5) and ZC15,400 (SEQ ID NO:6). Thermocycler conditions were as follows; one cycle at 94° C. for 1 min. 30 sec.; 30 cycles at 94° C. for 10 sec., 60° C. for 20 sec., 72° C. for 30 sec; one cycle at 72°C. for 7 min.; followed by a 4° C. hold. The library was deconvoluted down to a positive pool of 250 clones. E. coli DH10B cells (Gibco BRL, Rockville, Md.) were transformed with this pool by electroporation following manufacturer's protocol. The transformants were plated out to individual colonies and screened by PCR using the previously described thermocycler conditions. A positive clone was identified and sequenced.

Additional 5′ sequence was generated by PCR using as template a positively identified pool of an arrayed human testis cDNA/plasmid library. The pool was identified by PCR using primers ZC18,622 (SEQ ID NO:7) and ZC18,623 (SEQ ID NO:8). Thermocycler conditions were as described previously. PCR using ZC18,622 (SEQ ID NO:7) and the vector primer ZC14,063 (SEQ ID NO:9) was carried out. Thermocycler conditions were as follows; one cycle at 94° C. for 2min.; 5 cycles at 94° C. for 10 sec., 68° C. for 1.5 min.; 25 cycles at 94° C. for 10 sec., 58° C. for 20 sec., 72° C. for 2 min.; one cycle at 72° C. for 7 min.; followed by a 4° C. hold. The PCR product was gel extracted using the Qiagen II gel extraction kit (Qiagen, Valencia, Calif.) and protocol. Sequencing of the PCR fragment extended the zapop2 sequence. The full length zapop2 polynucleotide sequence is shown in SEQ ID NO:1 and its corresponding polypeptide sequence is shown in SEQ ID NO:2.

### Example 2

### Tissue Distribution

Northerns were performed using Human Multiple Tissue Blots (MTN1, MTN2 and MTN3) from Clontech (Palo Alto, Calif.) to determine the tissue distribution of human zapop2. A 171bp cDNA probe was obtained using the PCR. Oligonucleotides ZC15,399 (SEQ ID NO:5) and ZC15,400 (SEQ ID NO:6) were used as primers. Marathon cDNA from MCF-7 (breast adenocarcinoma) was used as a template. The probe was purified using a Gel Extraction Kit (Qiagen) according to manufacturer's instructions. The probe was radioactively labeled using a Rediprime II DNA labeling kit (Amersham, Arlington Heights, Ill.) according to the manufacturer's specifications. The probe was purified using a NUCTRAP™ push column (Stratagene Cloning Systems, La Jolla, Calif.). EXPRESSHYB™ (Clontech) solution was used for prehybridization and as a hybridizing solution for the Northern blots. Hybridization took place overnight at 55° C., using 2×10⁶ cpm/ml labeled probe. The blots were then washed in 2×SSC and 0.1% SDS at room temperature, then with 2×SSC and 0.1% SDS at 65° C., followed by a wash in 0.1×SSC and 0.1% SDS at 65° C. The blots were exposed 4 days to Biomax MS film (Kodak, Rochester, N.Y.).

Two major transcript sizes were observed in all tissues at approximately 1 kb and 1.4 kb. Signals were strongest in heart and skeletal muscle, and lesser in other tissues represented on the blots. Larger transcript sizes were seen in other tissues as well, including approximately 7 kb in placenta, lung, and colon and approximately 4 kb in skeletal muscle and bone marrow and could represent alternatively spliced messages or incompletely spliced RNA.

A RNA Master Dot Blot (Clontech) that contained RNAs from various tissues that were normalized to 8 housekeeping genes was also probed and hybridized as described above. Low level expression was seen in all tissues with high level expression seen in heart.

### Example 3

### Chromosomal Assignment and Placement of Zapop2

Zapop2 was mapped to chromosome 1 using the commercially available “GeneBridge 4 Radiation Hybrid Panel” (Research Genetics, Inc., Huntsville, Ala.). The GeneBridge 4 Radiation Hybrid Panel contains DNAs from each of 93 radiation hybrid clones, plus two control DNAs (the HFL donor and the A23 recipient). A publicly available WWW server (http://www-genome.wi.mit.edu/cgi-bin/contig/rhmapper.pl) allows mapping relative to the Whitehead Institute/MIT Center for Genome Research's radiation hybrid map of the human genome (the “WICGR” radiation hybrid map) which was constructed with the GeneBridge 4 Radiation Hybrid Panel.

For the mapping of Zapop2 with the “GeneBridge 4 RH Panel”, 20 μl reactions were set up in a 96-well microtiter plate (Stratagene, La Jolla, Calif.) and used in a “RoboCycler Gradient 96” thermal cycler (Stratagene). Each of the 95 PCR reactions consisted of 2 μl 10×KlenTaq PCR reaction buffer (CLONTECH Laboratories, Inc., Palo Alto, Calif.), 1.6 μl dNTPs mix (2.5 mM each, PERKIN-ELMER, Foster City, Calif.), 1 μl sense primer, ZC 18,706, (SEQ ID NO:10), 1 μl antisense primer, ZC 18,707, (SEQ ID NO:11), 2 μl “RediLoad” (Research Genetics, Inc., Huntsville, Ala.), 0.4 μl 50×Advantage KlenTaq Polymerase Mix (Clontech Laboratories, Inc.), 25 ng of DNA from an individual hybrid clone or control and ddH₂O for a total volume of 20 μl. The reactions were overlaid with an equal amount of mineral oil and sealed. The PCR cycler conditions were as follows: an initial 1 cycle 5 minute denaturation at 95° C., 40 cycles of a 1 minute denaturation at 95° C., 1 minute annealing at 70° C. and 1.5 minute extension at 72° C., followed by a final 1 cycle extension of 7 minutes at 72° C. The reactions were separated by electrophoresis on a 2% agarose gel (Life Technologies, Gaithersburg, Md.).

The results showed that Zapop2 maps 2.12 cR_{—}3000 proximal to the framework marker NIB1364 on the chromosome 1 WICGR radiation hybrid map. The use of surrounding genes/markers positions Zapop2 in the 1p36 chromosomal region.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polynucleotide that encodes a polypeptide comprising a sequence of amino acid residues that is at least 90% identical to an amino acid sequence selected from the group consisting of:
(a) the amino acid sequence as shown in SEQ ID NO:2 from amino acid number 476 (Cys), to amino acid number 599 (Val); and
(b) the amino acid sequence as shown in SEQ ID NO:2 from amino acid number 1 (Met), to amino acid number 599 (Val).

2. An isolated polynucleotide molecule according to claim 1, wherein the polynucleotide is selected from the group consisting of:
(a) polynucleotide molecules comprising a nucleotide sequence as shown in SEQ ID NO:1 from nucleotide 1507 to nucleotide 1879; and
(b) polynucleotide molecules comprising a nucleotide sequence as shown in SEQ ID NO:1 from nucleotide 83 to nucleotide 1879; and
(b) polynucleotide molecules complementary to (a).

3. An isolated polynucleotide sequence according to claim 1, wherein the polynucleotide comprises nucleotide 1 to nucleotide 1797 of SEQ ID NO:3.

4. An isolated polynucleotide according to claim 1, wherein the polypeptide comprises a sequence of amino acid residues selected from the group consisting of:
(a) the amino acid sequence as shown in SEQ ID NO:2 from amino acid number 476 (Cys), to amino acid number 599 (Val); and
(b) the amino acid sequence as shown in SEQ ID NO:2 from amino acid number 1 (Met), to amino acid number 599 (Val).

5. The isolated polynucleotide molecule of claim 1, wherein the polynucleotide further encodes a polypeptide that contains at least one RING finger domain or at least one ankyrin repeat motif.

6. The isolated polynucleotide molecule of claim 1, wherein the polynucleotide further encodes a polypeptide that contains at least one RING finger domain and at least one ankyrin repeat motif.

7. An expression vector comprising the following operably linked elements:
a transcription promoter;
a DNA segment encoding a polypeptide having an amino acid sequence as shown in SEQ ID NO:2 from amino acid number 1 (Met), to amino acid number 599 (Val); and
a transcription terminator,
wherein the promoter is operably linked to the DNA segment, and the DNA segment is operably linked to the transcription terminator.

8. An expression vector according to claim 7, further comprising a secretory signal sequence operably linked to the DNA segment.

9. A cultured cell into which has been introduced an expression vector according to claim 7, wherein the cell expresses the polypeptide encoded by the DNA segment.

10. A DNA construct encoding a fusion protein, the DNA construct comprising:
a first DNA segment encoding a polypeptide comprising a sequence of amino acid residues selected from the group consisting of:
(a) the amino acid sequence as shown in SEQ ID NO:2 from amino acid number 1 (Met), to amino acid number 74 (Pro);
(b) the amino acid sequence as shown in SEQ ID NO:2 from amino acid number 75 (Glu), to amino acid number 376 (Leu);
(c) the amino acid sequence as shown in SEQ ID NO:2 from amino acid number 476 (Cys), to amino acid number 510 (Cys);
(d) the amino acid sequence as shown in SEQ ID NO:2 from amino acid number 555 (Cys), to amino acid number 587 (Cys); and
at least one other DNA segment encoding an additional polypeptide,
wherein the first and other DNA segments are connected in-frame; and
wherein the first and other DNA segments encode the fusion protein.

11. An expression vector comprising the following operably linked elements:
a transcription promoter;
a DNA construct encoding a fusion protein according to claim 10; and
a transcription terminator,
wherein the promoter is operably linked to the DNA construct, and the DNA construct is operably linked to the transcription terminator.

12. A cultured cell comprising an expression vector according to claim 11, wherein the cell expresses a polypeptide encoded by the DNA construct.

13. A method of producing a fusion protein comprising:
culturing a cell according to claim 12; and
isolating the polypeptide produced by the cell.

14. An isolated polypeptide comprising a sequence of amino acid residues that is at least 90% identical to an amino acid sequence selected from the group consisting of:
(a) the amino acid sequence as shown in SEQ ID NO:2 from amino acid number 476 (Cys), to amino acid number 599 (Val); and
(b) the amino acid sequence as shown in SEQ ID NO:2 from amino acid number 1 (Met), to amino acid number 599 (Val).

15. An isolated polypeptide according to claim 14, wherein the polypeptide comprises a sequence of amino acid residues selected from the group consisting of:
(a) the amino acid sequence as shown in SEQ ID NO:2 from amino acid number 476 (Cys), to amino acid number 599 (Val); and
(b) the amino acid sequence as shown in SEQ ID NO:2 from amino acid number 1 (Met), to amino acid number 599 (Val).

16. The isolated polypeptide of claim 14, wherein the polypeptide further contains at least one RING finger domain or at least one ankyrin repeat motif.

17. The isolated polypeptide of claim 14, wherein the polypeptide further contains at least one RING finger domain and at least one ankyrin repeat motif.

18. A method of producing a polypeptide comprising:
culturing a cell according to claim 9; and
isolating the polypeptide produced by the cell.

19. A method of producing an antibody to a polypeptide comprising:
inoculating an animal with a polypeptide selected from the group consisting of:
(a) a polypeptide according to claim 15;
(b) the amino acid sequence as shown in SEQ ID NO:2 from amino acid number 1 (Met), to amino acid number 74 (Pro);
(c) the amino acid sequence as shown in SEQ ID NO:2 from amino acid number 75 (Glu), to amino acid number 376 (Leu);
(d) the amino acid sequence as shown in SEQ ID NO:2 from amino acid number 476 (Cys), to amino acid number 510 (Cys);
(e) the amino acid sequence as shown in SEQ ID NO:2 from amino acid number 555 (Cys), to amino acid number 587 (Cys); and
wherein the polypeptide elicits an immune response in the animal to produce the antibody; and
isolating the antibody from the animal.

20. An antibody produced by the method of claim 19, which specifically binds to a polypeptide of SEQ ID NO:2.

21. The antibody of claim 20, wherein the antibody is a monoclonal antibody.

22. An antibody that specifically binds to a polypeptide of claim 15.

23. A method of detecting, in a test sample, the presence of an agonist of zapop2 protein activity, comprising:
transfecting a zapop2-expressing cell, with a reporter gene construct that is responsive to a zapop2-stimulated cellular pathway; and
adding a test sample; and
comparing levels of response in the presence and absence of the test sample, by a biological or biochemical assay; and
determining from the comparison, the presence of the agonist of zapop2 activity in the test sample.

24. A method for detecting a genetic abnormality in a patient, comprising:
obtaining a genetic sample from a patient;
producing a first reaction product by incubating the genetic sample with a polynucleotide comprising at least 14 contiguous nucleotides of SEQ ID NO:1 or the complement of SEQ ID NO:1, under conditions wherein said polynucleotide will hybridize to complementary polynucleotide sequence;
visualizing the first reaction product; and
comparing said first reaction product to a control reaction product from a wild type patient, wherein a difference between said first reaction product and said control reaction product is indicative of a genetic abnormality in the patient.

25. A method for detecting a cancer in a patient, comprising:
obtaining a tissue or biological sample from a patient;
incubating the tissue or biological sample with an antibody of claim 22 under conditions wherein the antibody binds to its complementary polypeptide in the tissue or biological sample;
visualizing the antibody bound in the tissue or biological sample; and
comparing levels of antibody bound in the tissue or biological sample from the patient to a normal control tissue or biological sample,
wherein an increase or decrease in the level of antibody bound to the patient tissue or biological sample relative to the normal control tissue or biological sample is indicative of a cancer in the patient.

26. A method for detecting a cancer in a patient, comprising:
obtaining a tissue or biological sample from a patient;
labeling a polynucleotide comprising at least 14 contiguous nucleotides of SEQ ID NO:1 or the complement of SEQ ID NO:1;
incubating the tissue or biological sample with under conditions wherein the polynucleotide will hybridize to complementary polynucleotide sequence;
visualizing the labeled polynucleotide in the tissue or biological sample; and
comparing the level of labeled polynucleotide hybridization in the tissue or biological sample from the patient to a normal control tissue or biological sample,
wherein an increase or decrease in the labeled polynucleotide hybridization to the patient tissue or biological sample relative to the normal control tissue or biological sample is indicative of a cancer in the patient.
